(19) United States
(12) Patent Application Publication
(10) Pub. No.: US 2026/0001908 A1
(43) Pub. Date: Jan. 1, 2026

(54) METHOD FOR PURIFYING AN ANTIBODY

(71) Applicant: ASAHI KASEI LIFE SCIENCE CORPORATION, Tokyo (JP)

(72) Inventors: Junji HIDAKA, Tokyo (JP); Hiroki TANIGUCHI, Tokyo (JP); Akika FUTAMURA, Tokyo (JP)

(73) Assignee: ASAHI KASEI LIFE SCIENCE CORPORATION, Tokyo (JP)

(21) Appl. No.: 19/105,523

(22) PCT Filed: Sep. 11, 2023
(86) PCT No.: PCT/JP2023/033020

§ 371 Date: Feb. 21, 2025

(30) Foreign Application Priority Data
Sep. 12, 2022 (JP) 2022-144658

(51) Int. Cl. C07K 1/34; C07K 16/00

(52) U.S. Cl. CPC C07K 1/34; C07K 16/00

(57) ABSTRACT

Provided is a method for purifying an antibody, comprising: providing a solution containing the antibody; and filtering the solution using a porous membrane comprising regenerated cellulose to purify the antibody. Also provided is a method for purifying an antibody, comprising: providing a solution containing the antibody; and filtering the solution using a porous membrane comprising regenerated cellulose to purify the antibody, wherein (A) Tr−Tm of the antibody in hydrophobic interaction chromatography is 15 minutes or more, and/or (B) a k value of the antibody in hydrophobic interaction chromatography is 19 or more.

### TECHNICAL FIELD

The present invention relates to a method for purifying an antibody.

### BACKGROUND ART

For plasma fractionation products derived from human blood and biological preparations such as biopharmaceuticals, a virus removal/inactivation step is introduced in their production processes as a measure for improving safety against viruses. In particular, a virus removal method by filtration with a porous hollow fiber membrane is an effective method that can reduce viruses without denaturing useful proteins (see, for example, Patent Literatures 1 to 3).

### CITATION LIST

### Patent Literature

Patent Literature 1: International Publication No. WO 2015/156401
Patent Literature 2: International Publication No. WO 2017/170874
Patent Literature 3: International Publication No. WO 2022/118943

### SUMMARY OF INVENTION

### Technical Problem

Useful proteins for use in biological preparations include antibodies. Multispecific antibodies having specificity for a plurality of antigens by having a plurality of antigen binding sites have received attention in recent years. Some antibodies, including multispecific antibodies, easily clog filters and are thus difficult to filterate. Accordingly, an object of the present invention is to provide a method for purifying an antibody which is capable of purifying an antibody that is difficult to filtrate.

### Solution to Problem

[1] A method for purifying an antibody according to an embodiment comprises: providing a solution containing the antibody; and filtering the solution using a porous membrane to purify the antibody, the porous membrane comprising regenerated cellulose, wherein in the solution containing the antibody, a particle size distribution of the antibody includes a range of 22.0 nm or larger. In this context, the particle size distribution may be measured by a dynamic light scattering method.

[2] In the method for purifying an antibody according to [1], the porous membrane comprising regenerated cellulose may be a porous hollow fiber membrane.

[3] In the porous membrane comprising regenerated cellulose according to [1] or [2], an elastic limit pressure may be 200 kPa or higher.

[4] In the method for purifying an antibody according to any of [1] to [3], in the solution containing the antibody, the particle size distribution of the antibody may include a range of 24.0 nm or larger.

[5] In the method for purifying an antibody according to any of [1] to [4], the antibody may comprise aggregates of the antibody.

[6] In the method for purifying an antibody according to any of [1] to [5], the antibody may be an antibody capable of binding to two or more different antigens.

[7] In the method for purifying an antibody according to any of [1] to [6], the antibody may be a monoclonal antibody.

[8] In the method for purifying an antibody according to any of [1] to [7], the antibody may be contained in an antibody-drug conjugate.

[9] In the method for purifying an antibody according to any of [2] to [8], a ratio (R/t) of an inner diameter (R) to a membrane thickness (t) of the porous hollow fiber membrane may be 8.4 or less.

[10] In the method for purifying an antibody according to any of [2] to [9], a membrane thickness (t) of the porous hollow fiber membrane may be in a range of 20 μm or larger and 70 μm or smaller.

[11] In the method for purifying an antibody according to any of [1] to [10], the regenerated cellulose may be regenerated cellulose produced by a cuprammonium method.

[12] In the method for purifying an antibody according to any of [2] to [11], a pore size on an inner surface of the porous hollow fiber membrane may be larger than that on an outer surface thereof.

[13] In the method for purifying an antibody according to any of [2] to [12], the porous hollow fiber membrane may have a gradient structure where a pore size becomes smaller from an inner surface side toward an outer surface side.

[14] In the method for purifying an antibody according to any of [2] to [13], a water permeability of the porous hollow fiber membrane at a filtration pressure of 27 kPa and 37° C. may be 10 L/(m2·hr) or more and 50 L/(m2·hr) or less.

[15] In the method for purifying an antibody according to any of [2] to [14], a bubble point of the porous hollow fiber membrane may be 1.2 MPa or more.

[16] In the method for purifying an antibody according to any of [1] to [15], a virus may be removed in purifying the antibody.

[17] In the method for purifying an antibody according to any of [2] to [16], a parvovirus removal rate (LRV) of the porous hollow fiber membrane may be 4.0 or more.

[18] In the method for purifying an antibody according to any of [1] to [17], pH of the solution containing the antibody may be 4.5 or higher and 10.0 or lower.

[19] In the method for purifying an antibody according to any of [1] to [18], a salt concentration of the solution containing the antibody may be 0 mmol/L or higher and 1000 mmol/L or lower.

[20] In the method for purifying an antibody according to any of [1] to [19], an electric conductivity of the solution containing the antibody may be 0 mS/cm or more and 100 mS/cm or less.

[21] A method for purifying an antibody according to an embodiment comprises: providing a solution containing the antibody; and filtering the solution using a porous membrane to purify the antibody, the porous membrane comprising regenerated cellulose wherein (A) Tr−Tm of the antibody in hydrophobic interaction chromatography is 15 minutes or more, and/or (B) a k value of the antibody in hydrophobic interaction chromatography is 19 or more. In this context, Tr represents a time required for an antibody to pass through a hydrophobic interaction chromatography column and reach a detector in a hydrophobic interaction chromatography apparatus. Tm represents a time required for an antibody to reach a detector in a hydrophobic interaction chromatography apparatus from which a hydrophobic interaction chromatography column has been removed. The k value is provided according to the expression given below. T0 represents a time required for an antibody to reach a detector in a hydrophobic interaction chromatography apparatus, the antibody going through a hydrophobic interaction chromatography column without interacting with the column.

$$K = ( Tr - T 0 ) / ( T 0 - Tm )$$

[22] In the method for purifying an antibody according to [21], the porous membrane may be a porous hollow fiber membrane.

[23] In the method for purifying an antibody according to [21] or [22], an elastic limit pressure of the porous membrane comprising regenerated cellulose may be 200 kPa or higher.

[24] In the method for purifying an antibody according to any of [21] to [23], the Tr−Tm may be 16 minutes or more, and/or the k value may be 20 or more.

[25] In the method for purifying an antibody according to any of [21] to [24], the antibody may be an antibody capable of binding to two or more different antigens.

[26] In the method for purifying an antibody according to any of [21] to [25], the antibody may be a monoclonal antibody.

[27] In the method for purifying an antibody according to any of [21] to [26], the antibody may be contained in an antibody-drug conjugate.

[28] In the method for purifying an antibody according to any of [22] to [27], a ratio (R/t) of an inner diameter (R) to a membrane thickness (t) of the porous hollow fiber membrane may be 8.4 or less.

[29] In the method for purifying an antibody according to any of [22] to [28], a membrane thickness (t) of the porous hollow fiber membrane may be in a range of 20 μm or larger and 70 μm or smaller.

[30] In the method for purifying an antibody according to any of [21] to [29], the regenerated cellulose may be regenerated cellulose produced by a cuprammonium method.

[31] In the method for purifying an antibody according to any of [22] to [30], a pore size on an inner surface of the porous hollow fiber membrane may be larger than that on an outer surface thereof.

[32] In the method for purifying an antibody according to any of [22] to [31], the porous hollow fiber membrane may have a gradient structure where a pore size becomes smaller from an inner surface side toward an outer surface side.

[33] In the method for purifying an antibody according to any of [22] to [32], a water permeability of the porous hollow fiber membrane at a filtration pressure of 27 kPa and 37° C. may be 10 L/(m2·hr) or more and 50 L/(m2·hr) or less.

[34] In the method for purifying an antibody according to any of [22] to [33], a bubble point of the porous hollow fiber membrane may be 1.2 MPa or more.

[35] In the method for purifying an antibody according to any of [22] to [34], a virus may be removed in purifying the antibody.

[36] In the method for purifying an antibody according to any of [21] to [35], a parvovirus removal rate (LRV) of the porous hollow fiber membrane may be 4.0 or more.

[37] In the method for purifying an antibody according to any of [21] to [36], pH of the solution containing the antibody may be 4.5 or higher and 10.0 or lower.

[38] In the method for purifying an antibody according to any of [21] to [37], a salt concentration of the solution containing the antibody may be 0 mmol/L or higher and 1000 mmol/L or lower.

[39] In the method for purifying an antibody according to any of [21] to [38], an electric conductivity of the solution containing the antibody may be 0 mS/cm or more and 100 mS/cm or less.

### Advantageous Effect of Invention

The present invention can provide a method for purifying an antibody which is capable of purifying an antibody that is difficult to filtrate.

### BRIEF DESCRIPTION OF DRAWINGS

FIG. 1 is a schematic view of a membrane cross section of a porous hollow fiber membrane according to an embodiment. The relationship between an inner diameter (R) and a membrane thickness (t) of the porous hollow fiber membrane is shown.

FIG. 2 is a graph showing a particle size distribution of an antibody.

FIG. 3 is a graph showing a retention time of an antibody on a hydrophobic interaction chromatography column.

FIG. 4 is a graph showing a filtration rate of a solution containing an antibody.

FIG. 5 is a graph showing a filtration rate of a solution containing an antibody.

FIG. 6 is a graph showing a filtration rate of a solution containing an antibody.

FIG. 7 is a graph showing a filtration rate of a solution containing an antibody.

FIG. 8 is a graph showing a filtration rate of a solution containing an antibody.

FIG. 9 is a graph showing a filtration rate of a solution containing an antibody.

FIG. 10 is a graph showing a filtration rate of a solution containing an antibody.

FIG. 11 is a graph showing a filtration rate of a solution containing an antibody.

FIG. 12 is a graph showing a filtration rate of a solution containing an antibody.

FIG. 13 is a graph showing a filtration rate of a solution containing an antibody.

FIG. 14 is a graph showing a particle size distribution of an antibody.

FIG. 15 is a graph showing a filtration rate of a solution containing an antibody.

### DESCRIPTION OF EMBODIMENTS

Hereinafter, the present invention will be described in detail with reference to a specific mode for carrying out the present invention (hereinafter, referred to as the “present embodiment”). However, the present invention is not limited to the present embodiment given below, and may be carried out in any mode without departing from the spirit of the present invention.

A method for purifying an antibody according to the present embodiment comprises: providing a solution containing the antibody; and filtering the solution using a porous membrane to purify the antibody, the porous membrane comprising regenerated cellulose. Examples of the form of the porous membrane specifically include porous hollow fiber membranes, porous flat membranes, nonwoven fabrics, and woven fabrics, any of which may be used. The elastic limit pressure of the porous membrane may be 200 kPa or higher.

In the solution containing the antibody, the particle size of the antibody may assume a distribution. The particle size distribution of the antibody may be measured by a dynamic light scattering (DLS) method. The details of the dynamic light scattering method will be described in Examples. The particle size distribution of the antibody obtained by the dynamic light scattering method described in Examples may include the range of 22.0 nm or larger, the range of 23.0 nm or larger, the range of 24.0 nm or larger, the range of 25.0 nm or larger, or the range of 26.0 nm or larger. The upper limit of the particle size distribution of the antibody may be 50 nm, 40 nm, 35 nm, or 30 nm. A particle that provides a large particle size in the particle size distribution may be a monomer of the antibody or may be aggregates of the antibody.

Examples of the index that indicates a feature of the antibody to be purified include the degree of hydrophobicity of the antibody. The degree of hydrophobicity of the antibody can be measured from the length of time Tr to pass through a hydrophobic interaction chromatography column and reach a detector. The time Tr becomes longer with increase in the hydrophobicity of the antibody. The time Tr may be influenced by the piping length of a hydrophobic interaction chromatography apparatus. The influence of the piping length can be canceled by subtracting, from Tr, time Tm at which a detection peak is obtained when the hydrophobic interaction chromatography column is removed. The value of Tr−Tm of the antibody contained in the solution to be purified in the present embodiment according to a measurement method, which will be described in detail in Examples, may be 15 minutes or more, 16 minutes or more, 17 minutes or more, 18 minutes or more, or 19 minutes or more. The value of Tr−Tm of the antibody may be 40 minutes or less, 35 minutes or less, 30 minutes or less, or 25 minutes or less.

A retention coefficient k value, which will be described in detail in Examples, can be used as an index that indicates the degree of hydrophobicity of the antibody without being influenced by the piping length of a chromatography apparatus or measurement conditions. The k value of the antibody contained in the solution to be purified in the present embodiment according to an analysis method, which will be described in Examples, may be 19 or more, 19.5 or more, or 20 or more. The k value of the antibody may be 30 or less, or less, 28 or less, or 25 or less.

The solution containing the antibody may contain aggregates of the antibodies. The aggregates of the antibodies include a dimeric or higher-order antibody. In general, the aggregates of the antibodies have a larger charge than that of a monomer of the antibody. Also, in general, the aggregates of the antibodies have higher hydrophobicity than that of a monomer of the antibody. A higher salt concentration of the solution tends to enhance antibody-antibody hydrophobic interaction and facilitate forming an antibody associate or aggregates. A higher temperature of the solution tends to increase the number of antibody aggregates.

The antibody may be a human antibody or may be an antibody protein derived from a nonhuman mammal such as a bovine or a mouse. Alternatively, the antibody may be a chimeric antibody protein with human IgG, or a humanized antibody. The chimeric antibody with human IgG is an antibody having variable regions derived from a nonhuman organism such as a mouse and additionally having constant regions replaced with ones of a human-derived immunoglobulin. The humanized antibody is an antibody having variable regions with complementarity-determining regions (CDRs) derived from a nonhuman organism and additionally with framework regions (FRs) derived from a human. The humanization further reduces immunogenicity as compared with the chimeric antibody.

The class (isotype) and subclass of the antibody are not particularly limited. Antibodies are classified, depending on structural difference in constant regions, into five classes IgG, IgA, IgM, IgD, and IgE. However, the antibody to be purified with the porous hollow fiber membrane according to an embodiment may be of any of the five classes. In human antibodies, IgG has four subclasses from IgG1 to IgG4, and IgA has two subclasses IgA1 and IgA2. The antibody to be purified with the porous hollow fiber membrane according to an embodiment may be of any of these subclasses. An antibody-associated protein such as an Fc-fusion protein which is a protein bound with an Fc region may also be included in the antibody to be purified with the porous hollow fiber membrane according to an embodiment.

Antibodies can also be classified depending on an origin. The antibody to be purified with the porous hollow fiber membrane according to the embodiment may be a natural human antibody, a recombinant human antibody produced by a gene recombination technique, a monoclonal antibody, or a polyclonal antibody. The antibody may be derived from a plasma product or may be derived from a cell culture solution. In the case of obtaining the antibody by cell culture, animal cells or a microbe can be used as cells. The animal cells are not particularly limited by their type. Examples thereof include CHO cells, Sp2/0 cells, NS0 cells, Vero cells, and PER.C6 cells. The microbe is not particularly limited by its type. Examples thereof include E. coli and yeasts.

The antibody may be a multispecific antibody having specificity for a plurality of antigens. The multispecific antibody is also called next-generation antibody. Examples of the multispecific antibody include whole antibodies, antibodies having two or more VL and VH domains, DART® (MacroGenics, Inc.) molecules, antibody fragments such as Fab, Fv, dsFv, scFv, diabody, bispecific diabody (TandAb), and triabody, and covalently or noncovalently linked antibody fragments.

A multispecific antibody having specificity for two types of antigens is called bispecific antibody. The bispecific antibody can specifically bind to two different epitopes on one biological molecule. Alternatively, the bispecific antibody can specifically bind to epitopes of two different biological molecules.

Antigen binding domains of the bispecific antibody comprise, for example, two VH/VL units, and the first VH/VL unit specifically binds to a first epitope while the second VH/VL unit specifically binds to a second epitope. Each VH/VL unit comprises a heavy chain variable domain (VH) and a light chain variable domain (VL).

The VH/VL unit comprises at least one H chain hypervariable region (VH HVR) and at least one L chain hypervariable region (VL HVR). The VH/VL unit comprises, for example, one, two, or all three VH HVRs and one, two, or all three VL HVRs. The VH/VL unit may further comprise at least a portion of framework regions. The VH/VL unit may comprise three VH HVRs and three VL HVRs. In this case, the VH/VL unit comprises one, two, three, or all four H chain framework regions and one, two, three, or all four L chain framework regions.

A VH/VL unit further comprising at least a portion of heavy chain constant regions and/or at least a portion of light chain constant regions is also called half-antibody. The half-antibody comprises at least a portion of a single heavy chain variable region and at least a portion of a single light chain variable region. The half-antibody is also defined as a monovalent antigen binding polypeptide. The half-antibody may further comprise a constant domain.

The half-antibody comprises, for example, a portion of a heavy chain variable region sufficient for enabling an intramolecular disulfide bond to be formed with another half-antibody. The half-antibody comprises, for example, a hole mutation or a knob mutation that permits heterodimerization with another half-antibody having a complementary hole mutation or knob mutation. In this context, a mutation that introduces a protuberance (knob) to a polypeptide is called knob mutation. A mutation that introduces a cavity (hole) to a polypeptide is called hole mutation.

For example, a bispecific antibody that comprises two half-antibodies and binds to two antigens comprises a first half-antibody that binds to a first epitope but does not bind to a second epitope, and a second half-antibody that binds to the second epitope but does not bind to the first epitope.

The bispecific antibody is, for example, a knob-in-hole (KiH) antibody. The knob-in-hole means that two polypeptides are paired by introducing a protuberance (knob) to one of the two polypeptides and introducing a cavity (hole) to the other polypeptide, at an interface where the polypeptides interact with each other. The knob-in-hole is introduced to, for example, an Fc:Fc binding interface, a CL:CH1 interface, or a VH/VL interface, of the antibody. The bispecific antibody is produced, for example, by pairing two different heavy chains through knob-in-hole. Alternatively, the bispecific antibody is, for example, a CrossMab antibody. The CrossMab means that respective halves of two different antibodies are bound to each other.

The multispecific antibody may have a plurality of common L chains. Different L chains against a plurality of antigens are engineered to prepare common L chains, which are in turn co-expressed with a plurality of H chains different from each other so that an antibody that maintains the ability to bind to the plurality of antigens can be prepared even though having the common L chains.

The antibody generally tends to easily undergo aggregation or association when having high hydrophobicity. The multispecific antibody also easily undergoes aggregation or association when having high hydrophobicity. However, the multispecific antibody, even if its hydrophobicity is low, tends to easily undergo aggregation or association. Although not bound by any theory, the multispecific antibody has asymmetric charge distributions in antibody molecules because of having a plurality of different antigen binding sites. Hence, the charge interaction between the antibody molecules is strong, presumably easily undergoing aggregation or association.

Thus, in the solution containing the multispecific antibody, the particle size distribution of the antibody may include the range of 22.0 nm or larger. Tr−Tm of the multispecific antibody in hydrophobic interaction chromatography may be 15 minutes or more. The k value of the multispecific antibody may be 19 or more.

The antibody may be contained in an antibody-drug conjugate (ADC). In the antibody-drug conjugate, a drug is attached to the antibody. Examples of the drug to be attached to the antibody include low-molecular medicaments, for example, anticancer agents.

The solvent for use in the solution containing the antibody may be pure water or may be a buffer solution. Examples of the buffer solution include, but are not particularly limited to, buffer solutions in which at least any one of tris salt, acetate, Tween, sorbitol, maltose, glycine, arginine, lysine, histidine, sulfonate, phosphate, citrate, and sodium chloride is dissolved. The concentration of the salt contained in the buffer solution is not particularly limited as long as the antibody is soluble therein. The lower limit value of the salt concentration of the buffer solution depends on the type of the buffer solution and is, for example, 0 mmol/L or higher, 0.5 mmol/L or higher, 1.0 mmol/L or higher, 5.0 mmol/L or higher, 10.0 mmol/L or higher, 15.0 mmol/L or higher, or 25.0 mmol/L or higher. The upper limit value of the salt concentration of the buffer solution depends on the type of the buffer solution and is, for example, 1000 mmol/L or lower, 900 mmol/L or lower, 800 mmol/L or lower, 700 mmol/L or lower, 600 mmol/L or lower, 500 mmol/L or lower, 400 mmol/L or lower, 300 mmol/L or lower, or 200 mmol/L or lower.

The pH of the buffer solution is not particularly limited. The lower limit value of the pH of the buffer solution depends on the type of the buffer solution and is, for example, 4.5 or higher, 5.0 or higher, 5.5 or higher, or 6.0 or higher. The upper limit value of the pH of the buffer solution depends on the type of the buffer solution and is, for example, 10.0 or lower, 9.0 or lower, 8.0 or lower, 8.5 or lower, 8.0 or lower, 7.5 or lower, or 7.0 or lower.

The electric conductivity of the buffer solution is not particularly limited. The lower limit value of the electric conductivity of the buffer solution depends on the type of the buffer solution and is, for example, 0 mS/cm or more, 1 mS/cm or more, 2 mS/cm or more, 3 mS/cm or more, 4 mS/cm or more, or 5 mS/cm or more. The upper limit value of the electric conductivity of the buffer solution depends on the type of the buffer solution and is, for example, 100 mS/cm or less, 90 mS/cm or less, 80 mS/cm or less, 70 mS/cm or less, 60 mS/cm or less, 50 mS/cm or less, 40 mS/cm or less, 30 mS/cm or less, or 20 mS/cm or less.

The concentration of the antibody in the solution is not particularly limited as long as the antibody is dissolved in the solution. The lower limit value of the concentration of the antibody in the solution is, for example, 0.01 mg/mL or higher, 0.05 mg/mL or higher, 0.10 mg/mL or higher, 0.50 mg/mL or higher, 1.00 mg/ml or higher, or 5.00 mg/ml or higher. The upper limit value of the concentration of the antibody in the solution is, for example, 100 mg/mL or lower, 90 mg/mL or lower, 80 mg/mL or lower, 70 mg/mL or lower, 60 mg/mL or lower, 50 mg/mL or lower, 40 mg/mL or lower, 30 mg/mL or lower, 25 mg/ml or lower, or 20 mg/ml or lower.

The porous hollow fiber membrane according to the present embodiment is a hollow membrane having a porous structure containing many pores for the permeation or trapping of a substance. Although the porous hollow fiber membrane is not particularly limited by its shape, the porous hollow fiber can have a cylindrically continuous shape. In the present specification, a surface positioned on the inner side of the cylinder of the porous hollow fiber membrane is referred to as an inner surface, and a surface positioned on the outer side of the cylinder is referred to as an outer surface.

The porous membrane according to the present embodiment is not particularly limited as long as the porous membrane comprises regenerated cellulose. The regenerated cellulose is not particularly limited as long as cellulose is regenerated by the shaping of a dope of natural cellulose dissolved by chemical treatment, followed by another chemical treatment. Examples thereof can include regenerated cellulose obtained by a method of preparation from a cuprammonium cellulose solution (cuprammonium method) or a method of preparation by the alkaline saponification of cellulose acetate (saponification method).

The porous membrane according to the present embodiment may comprise a component other than the regenerated cellulose, and a portion of the regenerated cellulose may be modified. Examples thereof include regenerated cellulose modified by the esterification of a cellulose hydroxy group and partially cross-linked regenerated cellulose. The surface of the porous membrane may be coated with a polymer film. Examples of the polymer for coating include polyhydroxyethyl methacrylate, copolymers of 2-hydroxyethyl methacrylate and acrylamide, polymethoxyethyl acrylate, copolymers of 2-hydroxyethyl methacrylate and diethylaminoethyl methacrylate, copolymers of 2-methacryloyloxyethyl phosphorylcholine and n-butyl methacrylate, copolymers of 2-(N-3-sulfopropyl-N, N-dimethylammonium)ethyl methacrylate and n-butyl methacrylate, hydroxypropylcellulose, polyvinylpyrrolidone, and copolymers of vinylpyrrolidone and vinyl acetate.

The elastic limit pressure of a porous flat membrane is defined as a pressure at which change in filtration flow rate associated with pressure increase caused by pressurization with air from a surface side having a layer with an equivalent or larger pore size of the flat membrane as compared with the other surface deviates from linear change and a filtration flow rate in the case of applying again a smaller pressure to the flat membrane after the operation is no longer equivalent to the filtration flow rate before the operation. The deviation from linear change of the filtration flow rate of the flat membrane occurs due to change in pore size ascribable to the plastic deformation of the flat membrane. In this context, the filtration flow rate of the flat membrane can be calculated by dividing the weight of a liquid that has permeated the flat membrane in injecting the liquid to the flat membrane by pressurization, by a time elapsed from the start of filtration. For various examinations, filtration, and an integrity test such as a leak test in the production process of the flat membrane, it is preferred that the porous flat membrane should have no substantial change in virus removal properties and water permeation properties between before and after a test, and it is preferred that a pressure equal to or lower than the elastic limit should be selected as a pressure for use in the test. The elastic limit pressure of the porous flat membrane according to the present embodiment is measured in a wetted state of the porous flat membrane with water.

The elastic limit pressure of the porous hollow fiber membrane is defined as a pressure at which expansion observed from change in the outer diameter of the hollow fiber membrane associated with pressure increase caused by the pressurization of the hollow fiber membrane with air from the inner surface side deviates from linear change. The deviation of the expansion of the hollow fiber membrane from linear change occurs by the plastic deformation of the hollow fiber membrane. For various examinations, filtration, and an integrity test such as a leak test in the production process of the porous hollow fiber membrane, it is preferred that the porous hollow fiber membrane should have no substantial change in virus removal properties and water permeation properties between before and after a test, and it is preferred that a pressure equal to or lower than the elastic limit should be selected as a pressure for use in the test. The elastic limit pressure of the porous hollow fiber membrane according to the present embodiment is measured in a wetted state of the porous hollow fiber membrane with water.

The elastic limit pressure of the porous membrane is 200 kPa or higher and may be 210 kPa or higher, 220 kPa or higher, 230 kPa or higher, 240 kPa or higher, or 250 kPa or higher. Alternatively, the elastic limit pressure of the porous hollow fiber membrane may be 215 kPa or higher, 225 kPa or higher, 235 kPa or higher, 245 kPa or higher, 255 kPa or higher, 270 kPa or higher, or 280 kPa or higher. The upper limit value of the elastic limit pressure of the porous hollow fiber membrane is not particularly limited as long as the pressure can be actually applied. Examples thereof include 1000 kPa or lower, 900 kPa or lower, 800 kPa or lower, 700 kPa or lower, 600 kPa or lower, 500 kPa or lower, 450 kPa or lower, 400 kPa or lower, 350 kPa or lower, and 300 kPa or lower.

The porous membrane having an elastic limit pressure of 200 kPa or higher is capable of accelerating a filtration rate as compared with a membrane having a low elastic limit pressure. This porous membrane is also capable of increasing the quantity of filtration per unit time. Further, the porous membrane is capable of having a smaller membrane area because of its good filtration efficiency, and is capable of reducing production cost. In addition, the porous membrane having an elastic limit pressure of 200 kPa or higher is not necessarily required to undergo an evaluation test using gold colloid in an integrity test such as a leak test.

Use of the porous membrane comprising regenerated cellulose and having the elastic limit pressure of 200 kPa or higher allows for filtration of a solution containing an antibody heretofore considered difficult to filter due to clogging. The antibody having a particle size distribution including the range of 22.0 nm or larger, the antibody having Tr−Tm of 15 minutes or more in hydrophobic interaction chromatography, and the antibody having a k value of 19 or more, mentioned above, correspond to the antibody heretofore considered difficult to filter due to clogging. The porous membrane comprising regenerated cellulose and having the elastic limit pressure of 200 kPa or higher is capable of filtering the antibody having the particle size distribution including the range of 22.0 nm or larger, the antibody having Tr−Tm of 15 minutes or more in hydrophobic interaction chromatography, and the antibody having the k value of 19 or more, while suppressing clogging.

Setting a high transmembrane pressure difference at the time of filtration has an economic advantage of increasing a throughput per unit time as well as the advantage of enhancing virus trapping reliability because in a virus removal membrane, the physical binding force of a virus to the membrane is increased with increase in transmembrane pressure difference at the time of filtration. The transmembrane pressure difference is preferably set to approximately 75% or less of the elastic limit pressure of the porous membrane. Thus, the transmembrane pressure difference at the time of filtration is preferably 165 kPa or higher, 188 kPa or higher, or 225 kPa or higher. In another aspect, the transmembrane pressure difference at the time of filtration is, for example, 150 kPa or higher, 200 kPa or higher, or 250 kPa or higher. The upper limit value of the transmembrane pressure difference at the time of filtration is not particularly limited as long as the pressure can be actually applied. Examples thereof include 1000 kPa or lower, 900 kPa or lower, 800 kPa or lower, 700 kPa or lower, 600 kPa or lower, 500 kPa or lower, 450 kPa or lower, 400 kPa or lower, 350 kPa or lower, and 300 kPa or lower. The transmembrane pressure difference is regarded as being synonymous with a filtration pressure in low-pressure filtration under conditions where no particularly high filtration exhaust pressure is applied. As for an approach of controlling the transmembrane pressure difference, a filtration object may be pressure-fed such that a constant pressure is applied to the porous membrane, or a filtration object may be filtered at a fixed rate without exceeding the elastic limit pressure of the porous membrane.

In this context, the transmembrane pressure difference of the porous flat membrane refers to a pressure difference between a pressure on the fluid inflow side of the porous flat membrane and a pressure on the fluid outflow side of the porous flat membrane. Thus, the transmembrane pressure difference of the porous flat membrane is, for example, a value obtained by subtracting the pressure on the fluid outflow side of the porous flat membrane from the pressure on the fluid inflow side of the porous flat membrane.

The transmembrane pressure difference of the porous hollow fiber membrane refers to a pressure difference between a pressure on the inner surface side of the porous hollow fiber membrane and a pressure on the outer surface side of the porous hollow fiber membrane. Thus, the transmembrane pressure difference of the porous hollow fiber membrane is, for example, a value obtained by subtracting the pressure on the outer surface side of the porous hollow fiber membrane from the pressure on the inner surface side of the porous hollow fiber membrane.

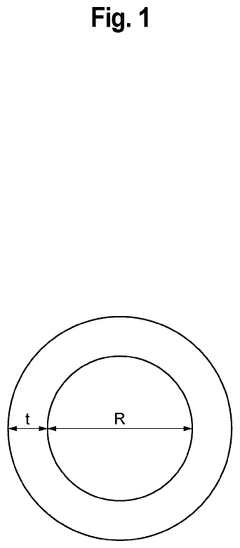

In the porous hollow fiber membrane according to the present embodiment, the ratio (R/t) of an inner diameter (R (μm)) to a membrane thickness (t (μm)) is preferably 8.4 or less. As shown in FIG. 1, the inner diameter (R) and the membrane thickness (t) are measured from a cross-sectional image of a round shape sliced from a hollow fiber in a dry state. The inner diameter is the inner surface diameter of the hollow fiber, and the membrane thickness is the vertical distance between the inner surface and the outer surface of the hollow fiber. Hereinafter, the inner diameter (R) and the membrane thickness (t) refer to values measured in a dry state unless otherwise specified.

The present inventors have found on the basis of the profile of the porous hollow fiber membrane that a porous hollow fiber membrane comprising regenerated cellulose suitable for a filtration object having a particle size of a little less than 20 nm to approximately 100 nm has a specific correlation between R/t, i.e., the ratio of the inner diameter (R) to the membrane thickness (t) of the porous hollow fiber membrane, and an elastic limit pressure. The upper limit value of R/t is preferably 8.4 or less from the viewpoint of achieving the elastic limit pressure of 200 kPa or higher in the porous hollow fiber membrane. R/t is more preferably in the range of 8.0 or less, further preferably in the range of 7.7 or less, according to the more preferred lower limit value of the elastic limit pressure mentioned above. The lower limit value of R/t is preferably 2.0 or more, i.e., the inner diameter is preferably two or more times the membrane thickness, from the viewpoint of stably producing a hollow fiber shape and satisfying the balance between a supply flow rate and a permeation flow rate as a hollow fiber filtration membrane.

The membrane thickness of the porous flat membrane is preferably in the range of 20 μm or larger and 100 μm or smaller. The membrane thickness is preferably 20 μm or larger from the viewpoint of conveniently designing a region for trapping a minute material through a sieve effect of the porous hollow fiber membrane. The membrane thickness is preferably 100 μm or smaller from the viewpoint of conveniently setting high permeation performance of the porous hollow fiber membrane. The membrane thickness of the porous hollow fiber membrane is more preferably in the range of 30 μm or larger and 80 μm or smaller, further preferably in the range of 40 μm or larger and 70 μm or smaller.

The membrane thickness of the porous hollow fiber membrane is preferably in the range of 20 μm or larger and 70 μm or smaller. The membrane thickness is preferably 20 μm or larger from the viewpoint of conveniently designing a region for trapping a minute material through a sieve effect of the porous hollow fiber membrane. The membrane thickness is preferably 70 μm or smaller from the viewpoint of conveniently setting high permeation performance of the porous hollow fiber membrane. The membrane thickness of the porous hollow fiber membrane is more preferably in the range of 30 μm or larger and 60 μm or smaller, further preferably in the range of 40 μm or larger and 50 μm or smaller.

At the time of antibody solution filtration, the pore size on a surface on the antibody solution inflow side of the porous flat membrane is preferably larger than that on a surface on the antibody solution outflow side thereof, from the viewpoint of achieving a high flow rate and suppressing the clogging of the porous flat membrane. It is more preferred to have a gradient structure where a pore size becomes smaller along the direction of travel of the antibody solution, and to further comprise a homogenous structure with less change in pore size for trapping fine particles to be removed, from the viewpoint of enhancing trapping performance for fine particles to be removed and suppressing the influence of clogging. In this context, the pore size is the size of a pore portion in an image obtained by observing, under an optical microscope or scanning electron microscope, both surfaces of the flat membrane or the cross section of a vertically cut surface of the flat membrane. The degree of a difference found by comparison is preferably clear enough to allow visual recognition of it from a microscopic image.

The pore size on the inner surface of the porous hollow fiber membrane is preferably larger than that on the outer surface thereof, from the viewpoint of achieving a high flow rate and suppressing the clogging of the porous hollow fiber membrane. It is more preferred to have a gradient structure where a pore size becomes smaller from the inner surface side toward the outer surface side, and to further comprise a homogenous structure with less change in pore size for trapping fine particles to be removed, from the viewpoint of enhancing trapping performance for fine particles to be removed and suppressing the influence of clogging. In this context, the pore size is the size of a pore portion in an image obtained by observing, under an optical microscope or scanning electron microscope, the inner surface or the outer surface of the membrane, or the cross section of a round shape sliced from the hollow fiber membrane. The degree of a difference found by comparison is preferably clear enough to allow visual recognition of it from a microscopic image.

The porous membrane comprises regenerated cellulose obtained from a cuprammonium method, from the viewpoint of achieving both a porous structure required for a virus removal membrane and an excellent hydrophilic feature. An exemplary method for producing the porous hollow fiber membrane according to the present embodiment by use of the cuprammonium method will be described below.

A spinning dope having a cellulose concentration of 6% by mass to 8% by mass, an ammonia concentration of 4% by mass to 5% by mass, and a copper concentration of 2% by mass to 3% by mass, which is obtained by dissolving cellulose in a cuprammonium solution, an internal coagulant which is an aqueous solution having an acetone concentration of 30% by mass to 50% by mass and an ammonia concentration of 0.5% by mass to 1.0% by mass, and an external coagulant which is an aqueous solution having an acetone concentration of 20% by mass to 40% by mass and an ammonia concentration of 0.2% by mass or lower, are first provided. The spinning dope may contain an inorganic salt such as sodium sulfate in a range on the order of 0.03% by mass to 0.1% by mass from the viewpoint of adjusting the microphase separation rate of the dope.

Next, the spinning dope is discharged at a rate of 2 mL/min to 5 mL/min from an annular double spinneret. At the same time, the internal coagulant is preferably discharged at a rate of 0.3 mL/min to 3.0 mL/min from a center spinning outlet disposed at a central portion of the annular double spinneret. For example, in order to obtain a hollow fiber inner diameter and a membrane thickness that achieve an elastic limit pressure exceeding 200 kPa, it is more preferred that the dope discharge rate should be 2.5 mL/min or more and 4 mL/min or less and the internal coagulant rate should be in the range of 0.3 mL/min or more and 1.6 mL/min or less, for adjusting the membrane thickness of the porous hollow fiber membrane to be produced to the preferred range of 20 μm to 70 μm. In a further preferred method, the internal coagulant rate is in the range of 0.3 mL/min or more and 1.4 mL/min or less. The spinning dope and the internal coagulant discharged from the annular double spinneret are immediately immersed in the external coagulant. After coagulation of the internal coagulant and the external coagulant, the membrane is taken up with a frame.

Examples of the immersion of the spinning dope and the internal coagulant in the external coagulant include a method of immersing the spinning dope and the internal coagulant in the external coagulant stored in a coagulating bath, a method of pursuing coagulation while causing the spinning dope and the internal coagulant, together with the external coagulant, to flow down and drop in a spinning funnel, and a method of also using a spinning funnel and using a U-shaped capillary. A method using a U-shaped capillary is preferred from the viewpoint of suppressing the extension of a coagulation process and thereby achieving a membrane structure having a high fine particle removal rate.

The temperature of the external coagulant is preferably controlled to a predetermined temperature selected from the range of 25° C. or higher and 45° C. or lower, from the viewpoint of forming the porous hollow fiber membrane as a membrane structure that stably achieves water permeation performance and virus removal performance mentioned later. The temperature is more preferably in the range of 30° C. or higher and 45° C. or lower, further preferably in the range of 35° C. or higher and 45° C. or lower.

The taken-up hollow fiber membrane is immersed in an aqueous solution containing 2% by mass to 10% by mass of dilute sulfuric acid and subsequently washed with pure water to regenerate cellulose. Further, water in the hollow fiber membrane is replaced with an organic solvent such as methanol or ethanol. Then, the hollow fiber membrane is dried under pressure under conditions of 30° C. to 60° C. and 5 kPa or lower while stretched by 1% to 8% with both ends of a hollow fiber membrane bundle fixed to obtain a porous hollow fiber membrane in a dry state.

In filtering the solution containing the antibody through the porous hollow fiber membrane, it is preferred to adopt a method of performing filtration in the direction of solution flow from the inner surface side toward the outer surface side of the hollow fiber (internal-pressure filtration method), for using the porous hollow fiber membrane so as to be able to effectively trap fine particles to be removed in the solution. The fine particles to be removed are, for example, a virus. Examples of the virus include parvovirus which is a small virus.

In the case of removing a virus such as parvovirus through the porous hollow fiber membrane, the water permeability of the porous hollow fiber membrane at a filtration pressure of 27 kPa and 37° C. is preferably 10 L/(m²·hr) or more and 50 L/(m²·hr) or less. The water permeability is a flow rate per unit time when water is filtered by an internal-pressure filtration method. A virus removal membrane designed so as to have a high water permeability can perform a virus removal step for biological preparations in a short time. Meanwhile, the water permeability is an index that indicates an average pore size of the whole porous hollow fiber membrane, and is designed according to the size of virus particles to be removed. Thus, the water permeability is preferably 10 L/(m²·hr) or more and 50 L/(m²·hr) or less, more preferably 15 L/(m²·hr) or more and 45 L/(m²·hr) or less, from the viewpoint of more reliably attaining trapping performance for parvovirus smaller than a diameter of 20 nm. In this context, the water permeability is defined under conditions of a filtration pressure of 27 kPa and 37° C. because these conditions are general measurement conditions for the water permeability in calculating the average pore size (nm) of the porous hollow fiber membrane in the art.

The water permeability of the porous hollow fiber membrane at a filtration pressure of 98 kPa and 25° C. is preferably 20 L/(m²·hr) or more and 100 L/(m²·hr) or less, more preferably 30 L/(m²·hr) or more and 85 L/(m² hr) or less.

The bubble point of the porous hollow fiber membrane is, for example, 1.2 MPa or more. In this context, the bubble point is an index that indicates the size of the largest pore of the porous hollow fiber membrane. The lower limit value of the bubble point is preferably 1.3 MPa or more, more preferably 1.4 MPa or more, further preferably 1.5 MPa or more, from the viewpoint of reliably trapping parvovirus smaller than a diameter of 20 nm. The upper limit value of the bubble point is preferably 2.4 MPa or less, more preferably 2.3 MPa or less, further preferably 2.2 MPa or less, from the viewpoint of achieving the water permeation properties mentioned above. The bubble point refers to a pressure at which the flow rate of a leaking gas is 2.4 mL/min in boosting a test module while immersing the test module in a fluorine-based liquid having a low surface tension, the test module being formed by sealing the porous hollow fiber membrane at one end to allow pressurization with air or nitrogen from the other end.

The virus removal properties of the porous membrane can be evaluated by a virus removal rate (LRV: logarithmic reduction value) which is a logarithmic value of a ratio between a stock solution containing a virus and a filtrate in 50% tissue culture infectious value (TCID₅₀/mL).

In the case of removing parvovirus through the porous hollow fiber membrane, the parvovirus removal rate is preferably 4.0 or more when a stock solution containing parvovirus at 6.0 TCID₅₀/mL or more and 8.0 TCID₅₀/mL or less is filtered with a transmembrane pressure difference of 196 kPa in an amount of 150 L/m². The parvovirus removal rate under the same conditions as above is more preferably 4.5 or more, further preferably 5.0 or more, in consideration of handling in a larger quantity of filtration and variation in filtration pressure. The LRV is preferably measured using a virus-containing protein solution described in (5-A) in a method for measuring LRV described in (5) as an exemplary measurement method in Examples mentioned later.

A longer filtration time using the porous membrane increases the quantity of filtration. The filtration time is, for example, 30 minutes or longer, 1 hour or longer, 3 hours or longer, or 6 hours or longer. The upper limit value of the filtration time is not particularly limited and is, for example, 7 days or shorter, 6 days or shorter, 5 days or shorter, 4 days or shorter, or 3 days or shorter.

The solution containing the antibody, before being purified using the porous membrane, may be purified or concentrated by another approach. Examples of another approach include methods using protein A carriers, affinity chromatography carriers, ion exchange chromatography carriers, hydrophobic interaction chromatography carriers, mixed mode chromatography carriers, hydroxyapatite, depth filters, ultrafiltration membranes, prefilters, and active carbon. The solution containing the antibody, after being purified using the porous hollow fiber membrane, may be purified or concentrated by the approach as described above. The method for purifying the solution containing the antibody using the porous hollow fiber membrane may be continuously carried out by linking this method to the approach as described above, or may be carried out independently from the method as described above.

### EXAMPLES

Hereinafter, the present invention will be described in more detail with reference to Examples. However, the present invention is not limited to Examples given below. The present invention may be carried out in any mode without departing from the spirit of the present invention.

### [Exemplary Measurement Method]

### (1) Method for Measuring Elastic Limit Pressure of Porous Hollow Fiber Membrane

A module for measurement is provided by sealing one end of one 50-mm long porous hollow membrane with a curable liquid resin such as urethane resin so as to prevent air leakage, and bonding and fixing the other end with a curable liquid resin such as urethane resin so as not to fill its hollow portion therewith while inserting the other end in a micro coupler (manufactured by Nitto Kohki Co., Ltd., MC-04PH). Aside from this, a pressurization apparatus equipped with micro coupler (manufactured by Nitto Kohki Co., Ltd., MC-10SM) is provided such that a pressure adjustment valve, a pressure gauge, and the micro coupler of the module for measurement can be connected to a piping for compressed air supply. The module for measurement is connected to the pressurization apparatus while immersed in water. The outer diameter of a hollow fiber is measured with a dimension measuring machine (manufactured by Keyence Corp., model LS-9006M) when a compressed air is supplied to the hollow portion by increasing the pressure at intervals of 20 kPa. The rate of change in outer diameter (%) at each measurement pressure is calculated according to the following expression, and a graph is drawn with the measurement pressure (kPa) on the X axis against the rate of change in outer diameter (%) on the Y axis.

$$Rate of change in outer diameter ( \% ) = ( D / {D}_{0} - 1 ) \times 100$$

wherein D: an outer diameter (μm) at each pressure, and D₀: an initial value of the outer diameter (μm) in a non-pressurized state

Next, a linear regression equation (Y=ax) which passes through an origin is determined using five measurement values at intervals of 20 kPa from 20 kPa to 100 kPa, and an equation (Y=ax+1) is introduced by adding 1, which means an extra 1% rate of change in outer diameter, to the right side of the equation. The line of the equation thus introduced is added to the graph. Of the pressures of a plot that does not exceed the rate of change in outer diameter on the line, the highest pressure is regarded as the elastic limit pressure of the module for measurement.

The test is conducted on six or more modules for measurements, and an average value therefrom is regarded as the elastic limit pressure of the porous hollow fiber membrane.

### (2) Method for Measuring Inner Diameter and Membrane Thickness

A cross-sectional slice of a porous hollow fiber membrane is prepared and photographed at 200× under a microscope (manufactured by Keyence Corp., model VHX-5000) to provide an image. The membrane thickness of the hollow fiber cross section on the image is measured at least 20 locations over the whole periphery, and an averaged value thereof is regarded as the measurement value of the membrane thickness. The inner diameter is calculated as the diameter of an approximated circle by determining the area of the hollow portion of the hollow fiber cross section on this image by image processing.

### (3) Method for Measuring Water Permeability of Porous Hollow Fiber Membrane

A module for measurement is provided by bundling 10 porous hollow fiber membranes, attaching a polyethylene tube connectable to a water permeation measuring machine to one end of the bundle with an adhesive, and sealing the other end of the hollow fiber while adjusting an effective length to 16 cm.

The water permeation measurement apparatus is equipped with a mechanism that discharges water at a fixed pressure from a conduit portion to which the polyethylene tube of the module for measurement is connectable, a mechanism capable of highly precisely determining the amount of the discharged liquid, a mechanism that measures a determination time of the amount of the discharged liquid, a bath in which the module for measurement is immersed, and a mechanism that controls the temperatures of discharged water and bath water.

The module for measurement is immersed in a water bath of 37° C. The conduit portion of the water permeation measuring machine is connected to the polyethylene tube of the module for measurement, and a time to inject 1 mL of water of 37° C. at 27 kPa is measured. A water permeability per m² of membrane area and hour (L/(m²·hr) is calculated from a filtration membrane area calculated on the basis of results of measuring the inner diameter (μm) of a porous hollow fiber membrane prepared under the same conditions as those for the porous hollow fiber membrane of the module for measurement, and the measurement value of the time to inject 1 mL of water.

The test is conducted on three or more modules for evaluation, and average value therefrom is regarded as the water permeability of the porous hollow fiber membrane.

### (4) Method for Measuring Bubble Point of Porous Hollow Fiber Membrane

A test module (effective length: 8 cm) is prepared by sealing one end of a porous hollow fiber membrane and fixing the other end to a metal coupler with a urethane resin to allow pressurization with air or nitrogen. A tube is attached to the test module, and 3M Novec 7200 Highly Functional Liquid™ (manufactured by 3M Japan Ltd.) is injected into the tube to immerse the porous hollow fiber membrane in the liquid.

A bubble point measurement apparatus is equipped with a pressure adjustment mechanism capable of pressurizing the inner surface side of the porous hollow fiber membrane via the metal coupler and gradually increasing the pressure, and a pressure display mechanism. The apparatus is equipped with a flow meter capable of measuring the flow rate of a gas flowing out of the tube of the test module.

The metal coupler portion of the test module is attached to the end portion of the pressurization mechanism, and a flow rate measurement mechanism line is attached to the end portion of the tube of the test module. The pressure (MPa) at which the flow rate of a gas leaked by gradual increase in pressure is 2.4 mL/min is detected. The test is conducted on three or more test modules, and an average value therefrom is regarded as the value of the bubble point.

### (5) Method for Measuring Virus LRV of Porous Hollow Fiber Membrane

The small membrane module described in FIG. 1 of Japanese Patent Laid-Open No. 2013-17990 is prepared by use of the known technique so as to have a membrane area of 0.001 m².

A solution to be filtered is prepared by the method described below in (5-A) or (5-B).

(5-A) For the preparation of a virus-containing protein solution, an antibody solution is first obtained by diluting a polyclonal antibody (human IgG) (Venoglobulin-IH, manufactured by Benesis Corp.) with injectable water (Otsuka Pharmaceutical Co., Ltd.) such that an antibody concentration is 1 mg/mL. The salt concentration thereof is also adjusted to 0.1 mol/L using a 1 mol/L aqueous NaCl solution. The hydrogen ion index (pH) thereof is further adjusted to 4.0 using 0.1 mol/L HCl or 0.1 mol/L NaOH to prepare a protein solution. The obtained protein solution is supplemented with 1.0 vol % of porcine parvovirus (PPV, Veterinary Biological Product Association) and well stirred to obtain a virus-containing protein solution.

(5-B) 0.2% porcine parvovirus (PPV, type VR742, purchased from American Type Culture Collection (hereinafter, referred to as ATCC)) is added to an aqueous solution of pH 4.5 containing 0.02 mol/L acetic acid and 0.1 mol/L NaCl, and the resulting aqueous solution is used as a virus-containing solution.

According to a dead-end internal-pressure filtration method using the provided 0.001 m² small membrane module and the virus-containing protein solution or the virus-containing solution, the virus-containing protein solution (5-A) is filtered until the quantity of filtration reaches 150 L/m², and the virus-containing solution (5-B) is filtered until the quantity of filtration reaches 5 L/m², to obtain a filtrate. Here, the filtration pressure is selected depending on the elastic limit pressure of the porous hollow fiber membrane. A porous hollow fiber having an elastic limit pressure of less than 200 kPa is used with 98 kPa as an appropriate pressure, and a porous hollow fiber having an elastic limit pressure of more than 200 kPa is used with 196 kPa as an appropriate pressure.

Next, in order to measure a virus infectivity titer, a solution (hereinafter, referred to as 3% FBS/D-MEM) of Dulbecco's Modified Eagle Medium (1×), liquid+4.5 g/L D-Glucose+L-Glutamine-Sodium Pyruvate™ (manufactured by Life Technologies Corporation; hereinafter, referred to as D-MEM) containing 3% BenchMark Fetal Bovine Serum™ (manufactured by Gemini Bio-Products Inc.) inactivated by heating for 30 minutes in a water bath of 56° C. and 1% PENICILLIN STREPTOMYCIN SOL™ (manufactured by Life Technologies Corporation), is provided. A filtration stock solution and a filtrate are each collected and diluted 10-fold, 10²-fold, 10³-fold, 10⁴-fold, and 10⁵-fold with 3% FBS-D-MEM.

PK-13 cells (No. CRL-6489, purchased from ATCC) are diluted with 3% FBS/D-MEM to prepare a diluted cell suspension having a cell density of 2.0×10⁵ (cells/mL), which is then dispensed at 100 μL/well to all the wells of ten 96-well round-bottom cell culture plates. In addition, the filtration stock solution, its dilutions, the filtrate, and its dilutions are each dispensed at 100 μL/well in every 8 wells. Then, the cells are cultured at 37° C. for 10 days in a 5% carbon dioxide atmosphere.

The 50% tissue culture infectious dose (TCID₅₀) of the cells cultured for 10 days is measured by use of the red cell adhesion method (see Virus Experimental Studies, Introduction, ed. by Japanese National Institute of Health Student's Association, p. 173).

Specifically, the method involves diluting Banked Chicken Blood™ (manufactured by Nippon Bio-Test Laboratories, Inc.) 5-fold with a PBS(−) adjustment solution of Dulbecco's PBS(−) Powder™ (manufactured by Nissui Pharmaceutical Co., Ltd.), then centrifuging the dilution at 2500 rpm at 4° C. for 5 minutes, removing a supernatant by suction, diluting the resulting precipitate 200-fold again with the PBS(−) adjustment solution, dispensing the resulting dilution at 100 μL/well to all the wells of the cell culture plates, leaving the plates standing for 2 hours, and then observing the adsorption of red cells to cell tissue surface to evaluate virus infection. The degrees of virus infection of the filtration stock solution, the filtrate, and their respective dilutions are confirmed, and the infectivity titer (TCID₅₀/mL) is calculated according to the Spearman-Karber expression.

The logarithmic reduction value (LRV) of the virus is calculated according to LRV=log₁₀(C₀/C_{F}) wherein C₀ represents an infectivity titer (TCID₅₀/mL) of the filtration stock solution, and C_{F} represents an infectivity titer (TCID₅₀/mL) of the filtrate obtained by filtration through the virus removal membrane.

The filtration rate of the virus-containing protein solution through the 0.001 m² membrane module is calculated as the quantity of filtration per m² of membrane area and hour (L/(m²·hr)) by measuring a time to reach a quantity of filtration of 150 L/m².

### (6) Method for Measuring Water Permeability of 0.001 m² Membrane Module

A water permeability per m² of membrane area and hour (L/(m²·hr)) is calculated by providing a 0.001 m² membrane module, filtering pure water through an ultrafiltration membrane by internal pressure filtration in a dead-end system at a temperature of 25° C. and a transmembrane pressure difference of 98 kPa for 10 minutes, and weighing a filtrate.

### (7) Method for Measuring Gold Colloid LRV of 0.001 m² Membrane Module

A gold colloid solution is prepared as a filtration stock solution by diluting AGP-HA20™ (manufactured by Asahi Kasei Medical Co., Ltd.), which is a solution containing gold colloid having a particle size of approximately 20 nm, with injectable distilled water (manufactured by Otsuka Pharmaceutical Co., Ltd.) and an aqueous solution containing 0.27% by mass of SDS (sodium lauryl sulfate), and adjusting an absorbance at a wavelength of 526 nm to 1.00 measured with an ultraviolet-visible spectrophotometer (manufactured by Shimadzu Corp., model UV-2450).

A 0.001 m² membrane module is provided. Filtration is carried out using the provided gold colloid solution by internal pressure filtration in a dead-end system under conditions involving a temperature of 25° C., a transmembrane pressure difference of 25 kPa, and a quantity of filtration of 2 L/m², and a filtrate is sampled from 0.5 L/m² to 2.0 L/m².

The respective absorbances at a wavelength of 526 nm of the filtration stock solution and the filtrate are measured using an ultraviolet-visible spectrophotometer (manufactured by Shimadzu Corp., model UV-2450), and the logarithmic reduction value (LRV) of the gold colloid particles is calculated according to the expression LRV=log₁₀(A/B) wherein A represents the absorbance of the filtration stock solution, and B represents the absorbance of the filtrate.

### [Results of Measuring Physical Properties of Porous Hollow Fiber Membrane]

Table 1 shows results of measuring an elastic limit pressure, an inner diameter (R), a membrane thickness (t), a water permeability, and a bubble point as to a porous hollow fiber membrane-type virus filter (Planova S20N®, Asahi Kasei Corp.) made of regenerated cellulose by various measurement methods described above. Table 1 also shows results of preparing a 0.001 m² membrane module as a small membrane module with a membrane area of 0.001 m² similar to FIG. 1 of Japanese Patent Laid-Open No. 2013-17990 by the known technique using the porous hollow fiber membrane-type virus filter, and measuring virus LRV (using the virus-containing protein solution described in (5-A) of the exemplary measurement method), a water permeability, and gold colloid LRV.

In order to confirm no change in the performance of the porous hollow fiber membrane in conducting an integrity test, Table 1 further shows ratios in water permeability and gold colloid LRV between before and after pressurization, which indicate the difference of results of pressurizing the inner surface side of the porous hollow fiber membrane in the obtained 0.001 m² membrane module with air at 250 kPa for 10 minutes while filling the outer surface side region of the porous hollow fiber membrane with pure water, and then measuring the water permeability and the gold colloid LRV, from results of performing this measurement without the pressurization mentioned above.

Here, the inventors selected gold colloid removal performance evaluation as an alternative approach of virus removal performance evaluation, because a virus evaluation method has measurement limitations depending on a virus concentration of the solution used, and the virus solution is rich in noninfectious particles and the like; thus, for determining a subtle difference in membrane structure, it is preferred to evaluate removal properties for gold colloid particles.

TABLE 1

| | |
|---|---|
| Elastic limit pressure (kPa) | 360 |
| Inner diameter (R, μm) | 279.8 |
| Membrane thickness (t, μm) | 45 |
| Ratio of inner diameter to membrane thickness (R/t) | 6.2 |
| Water permeability (L/(m² · hr)) *1 | 30.8 |
| Bubble point (Mpa) | 1.89 |
| Parvovirus (PPV) LRV | >5.5 |
| Water permeability of 0.001 m² membrane module | 58.9 |
| (L/(m² · hr)) *2 | |
| Water permeability of 0.001 m² membrane module after | 64.9 |
| pressurization at 50 kPa for 10 minutes (L/(m² · hr)) *2 | |
| Change in water permeability by pressurization at 250 kPa for | 1.11 |
| 10 minutes (before/after ratio) | |
| Gold colloid LRV of 0.001 m² membrane module | 1.73 |
| Water permeability of 0.001 m² membrane module after | 1.71 |
| pressurization at 50 kPa for 10 minutes (L/(m² · hr)) | |
| Change in water permeability by pressurization at 250 kPa for | 0.99 |
| 10 minutes (before/after ratio) | |

*1 Filtration condition: 27 kPa and 37° C.,
*2 Filtration condition: 98 kPa

The filtration rate of the virus-containing protein solution through the porous hollow fiber membrane-type virus filter (Planova S20N®, Asahi Kasei Corp.) made of regenerated cellulose was 145 LMH.

### Example 1 and Comparative Example 1

### (1) Preparation of Culture Supernatant

A culture solution containing monoclonal antibody mAb A (pembrolizumab, recombinant humanized IgG4 monoclonal antibody, molecular weight: approximately 149000)-producing CHO cells was filtered through a filtration membrane (manufactured by Asahi Kasei Medical Co., Ltd., trade name BioOptimal® MF-SL) to obtain a culture supernatant containing impurities and the antibody. The culture supernatant contained 1.5 g/L monoclonal antibody mAb A as an antibody protein.

### (2) Preparation of Antibody Solution (Purification of Culture Supernatant)

In a commercially typical method, the culture supernatant obtained in (1) was purified by protein A affinity chromatography and ion exchange chromatography, and the purified solution was buffer-replaced with a 15 mmol/L acetate buffer solution (pH 5.0, 22 mS/cm) containing sodium chloride to prepare a solution containing the antibody mAb A. The pH of the buffer solution was measured using a pH meter HM-30R (manufactured by DKK-TOA Corp.). The electric conductivity of the buffer solution was measured using an electric conductivity meter CM-42X (manufactured by DKK-TOA Corp.). The pH and electric conductivity values of a buffer solution mentioned later were obtained using the same measurement apparatuses as above. The prepared antibody solution was analyzed with a spectrophotometer NanoDrop One (manufactured by Thermo Fisher Scientific Inc.) to measure an antibody concentration, which was consequently 15.4 mg/mL.

### (3) DLS Measurement

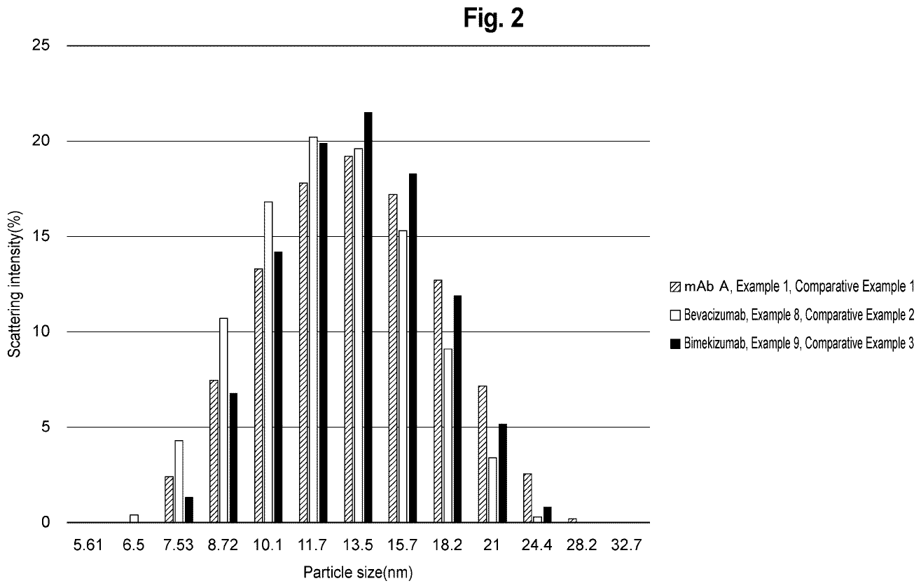

The solution containing the antibody mAb A prepared in (2) was injected to a polystyrene cuvette, and the particle size distribution of the antibody mAb A contained in the solution was measured under conditions given below using a dynamic light scattering (DLS) measurement apparatus Zetasizer Nano ZSP (manufactured by Spectris Co., Ltd.). The measurement was performed three times per sample, and an average value was obtained therefrom. The obtained average value is shown in FIG. 2. The largest value of the particle size was 28.2 nm, and the particle size with the largest scattering intensity was 13.5 nm. The particle size distribution of the antibody mAb A included the range of 22.0 nm or larger.

### Dynamic Light Scattering Measurement Method

Sample: Protein
Dispersion medium: Water
Temperature: 25° C., equilibration time: 15 seconds
Cell: Polystyrene cell ZEN0040
Measurement angle: 173° Backscatter (NIBS default)
Measurement time: Automated
The number of times of measurement: 3
Detailed setting: Automatic attenuator selection→Yes

### (4) HIC (Hydrophobic Interaction Chromatography) Measurement

The solution containing the antibody mAb A prepared in (2) was diluted with pure water such that the antibody concentration was 0.3 mg/mL. The time for the antibody to be detected in a detector from the start of supply to a column in an apparatus was measured under the following conditions using a high-performance liquid chromatograph Prominence (manufactured by Shimadzu Corp.).
Guard column: Filter assembly NPR (manufactured by Tosoh Corp.) with a built-in membrane filter (Fluoropore 0.45 μm FP-045, manufactured by Tosoh Corp.)
Main column: TSKgel Butyl-NPR (particle size: 2.5 μm, inner diameter: 4.6 mm, length: 10 cm, manufactured by Tosoh Corp.)
Column temperature: 35° C.
Flow rate: 1.0 mL/min
Injection volume: 100 μL
Detection wavelength: 280 nm
Buffer A: 20 mmol/L phosphate buffer solution+1.5 mol/L ammonium sulfate (pH 7.0)
Buffer B: 20 mmol/L phosphate buffer solution (pH 7.0)
Gradient: The proportion of the buffer B is elevated at a given rate from 0% at the start of measurement so as to become 100% 25 minutes later.

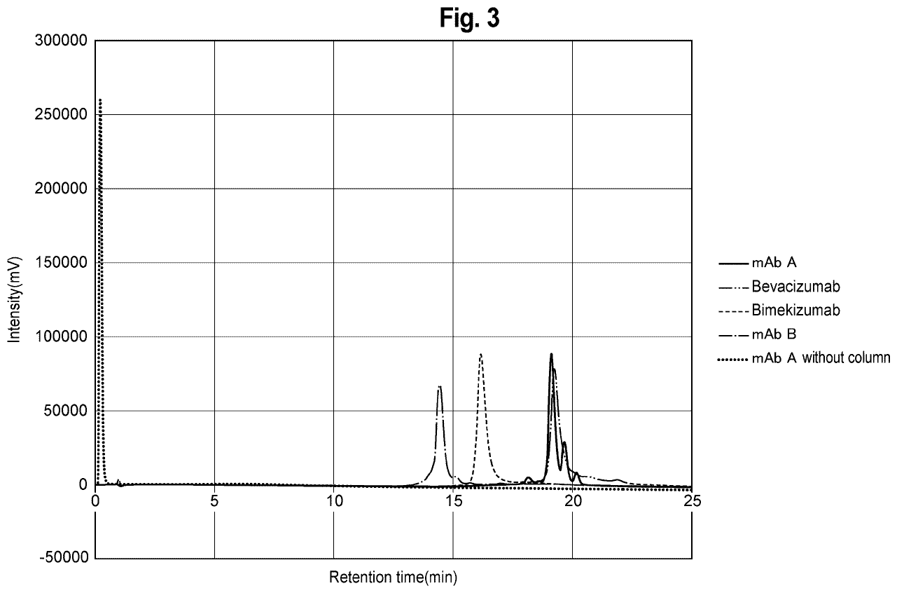

The time of the highest detection peak among detection peaks at retention times measured under the conditions described above was defined as Tr. As shown in FIG. 3, Tr of the antibody mAb A was 19.12 minutes.

The time for the antibody contained in the solution to pass through the hydrophobic interaction chromatography column and reach the detector is influenced by the piping length of the hydrophobic interaction chromatography apparatus. In order to study the influence, mAb A was analyzed by removing the guard column and the main column from the apparatus and attaching thereto Analytical Stainless Steel Union (manufactured by Shimadzu Corp., product No. 228-16004-13). As a result, a detection peak was obtained at 0.22 minutes. The time of this detection peak in the measurement without the column was defined as Tm. Tr−Tm of the antibody mAb A from which the influence of the piping length was removed was 18.90 minutes.

Since the time for the antibody contained in the solution to pass through the hydrophobic interaction chromatography column and reach the detector is influenced by the piping length of the hydrophobic interaction chromatography apparatus or measurement conditions, the following retention coefficient k value was defined as an index that indicated the degree of hydrophobicity of the antibody without being influenced thereby.

$$k = ( Tr - T 0 ) / ( T 0 - Tm )$$

Tr: Detection time (min) of the largest peak
T0: Detection time (min) of a peak in the case of injecting an antibody going through the column without interacting with the column.
Tm: Detection time (min) of a peak measured without the column

In hydrophobic chromatography, the detection time of a peak may vary depending on the salt concentration of buffer A. T0 can be represented by the detection time of a peak when the detection time of the peak is no longer changed even if the salt concentration of the buffer A is shifted from a high concentration to a low concentration. mAb B (nivolumab, recombinant human IgG4 monoclonal antibody) was analyzed by using 20 mmol/L phosphate buffer solution+3.0 mol/L sodium chloride (pH 7.0) and 20 mmol/L phosphate buffer solution+1.0 mol/L sodium chloride (pH 7.0) as the buffer A of (4) of Example 1. As a result, the detection time was 0.96 minutes in both the cases and was therefore used as T0. The k value was calculated on the basis of Tm, Tr, and T0 described above and was consequently 24.5 as shown in Table 4.

Thus, Tr−Tm of the antibody mAb A in hydrophobic interaction chromatography was 15 minutes or more, and the k value thereof was 19 or more.

### (5) Provision of Virus Removal Filter According to Comparative Examples

A hydrophilized PVDF hollow fiber membrane prepared in the same manner as the method described in and of Japanese Patent No. 5755136 was joined to a cartridge so as to have an effective membrane area of 0.000075 m² to prepare a virus removal filter according to Comparative Examples. Hereinafter, the virus removal filter according to Comparative Examples is also referred to as VF A.

### (6) Measurement of Flux Decay According to Comparative Examples

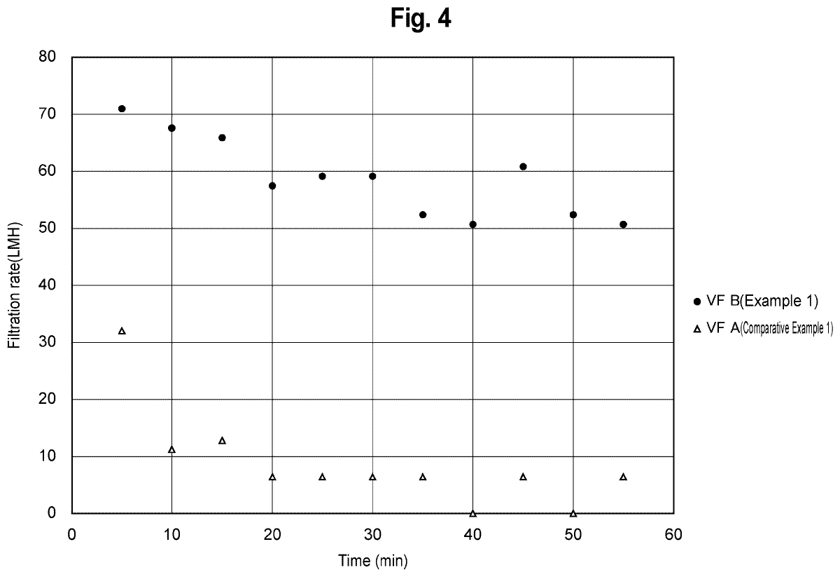

The solution containing the antibody mAb A prepared in (2) was injected at a fixed pressure of 196 kPa in a dead-end manner to the virus removal filter according to Comparative Examples prepared in (5). In this operation, an antibody solution flux rate per unit time per unit area of the virus removal filter according to Comparative Examples (LMH=the amount of the treated liquid (L)/virus removal membrane area (m²)/hour) was measured to obtain the results shown in FIG. 4. The following flux decay (%) was calculated as an index for the clogging of the virus removal filter.

$$Flux decay ( \% ) = ( V 120 - V 5 ) / V 5$$

V120: Antibody solution flux rate (LMH) 120 minutes after the start of filtration
V5: Antibody solution flux rate (LMH) 5 minutes after the start of filtration

V5 was 32. V120 was 3.2 or less because the antibody solution flux rate (LMH) was already 3.2 55 minutes after the start of filtration. Hence, the flux decay of VF B was 90% or more. Thus, even the virus removal filter capable of high-pressure filtration was drastically clogged and had large decrease in filtration rate when the solution containing the antibody mAb A was filtered therethrough.

### (7) Provision of Virus Removal Filter According to Examples

A hollow fiber-type virus filter (Planova S20N®, Asahi Kasei Corp.) made of regenerated cellulose was provided so as to have an effective membrane area of 0.000075 m² to prepare a virus removal filter according to Examples. Hereinafter, the virus removal filter according to Examples is also referred to as VF B.

### (8) Measurement of Flux Decay According to Examples

LMH of VF B was measured by the same method as in VA A using the solution containing the antibody mAb A to obtain the results shown in FIG. 4. The flux decay of VF B was 28.6% or more. Thus, VF B compared with VF A had a small flux decay at the time of filtration of mAb A and exerted stable filtration performance. Accordingly, VF B was found capable of purifying an antibody while suppressing clogging, wherein the particle size distribution of the antibody included the range of 22.0 nm or larger, and Tr−Tm and the k value of the antibody were 15 minutes or more and 19 or more, respectively, in hydrophobic interaction chromatography. Table 2 shows filtration conditions for virus removal filters used in the following Examples.

### Examples 2 to 4

A mAb A-containing solution prepared in the same manner as in (1) and (2) of Example 1 was buffer-replaced with a 15 mmol/L acetate buffer solution (pH 5.5, 20 mS/cm) containing sodium chloride in the same manner as in (2) of Example 1 to prepare a mAb A-containing solution according to Example 2 having an antibody concentration of 10.0 mg/mL, a mAb A-containing solution according to Example 3 having an antibody concentration of 30.0 mg/mL, and a mAb A-containing solution according to Example 4 having an antibody concentration of 50.0 mg/mL.

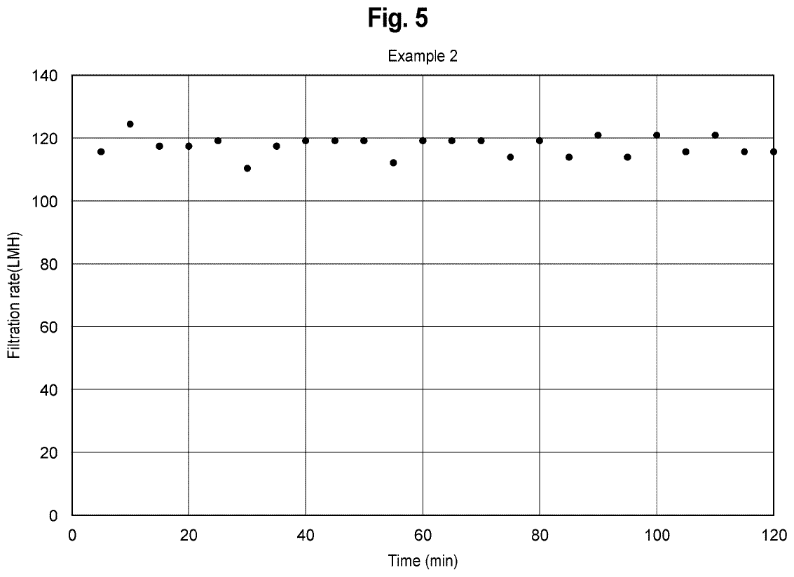

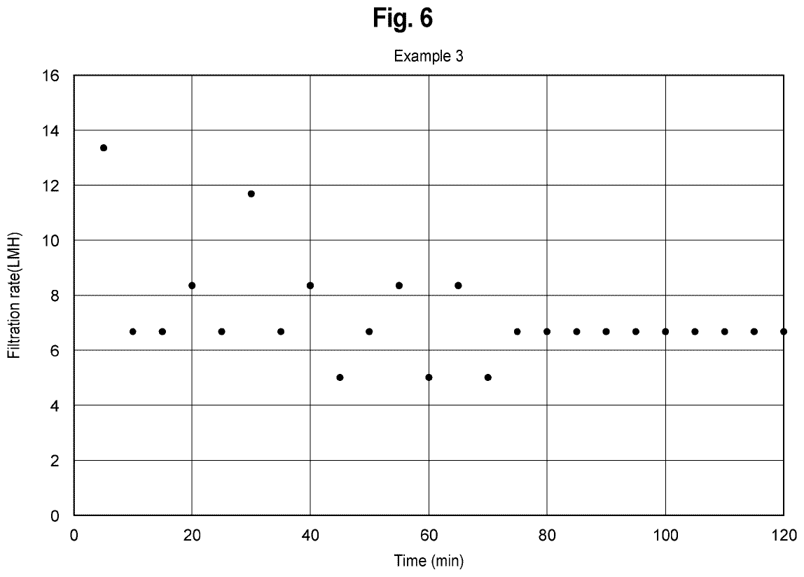

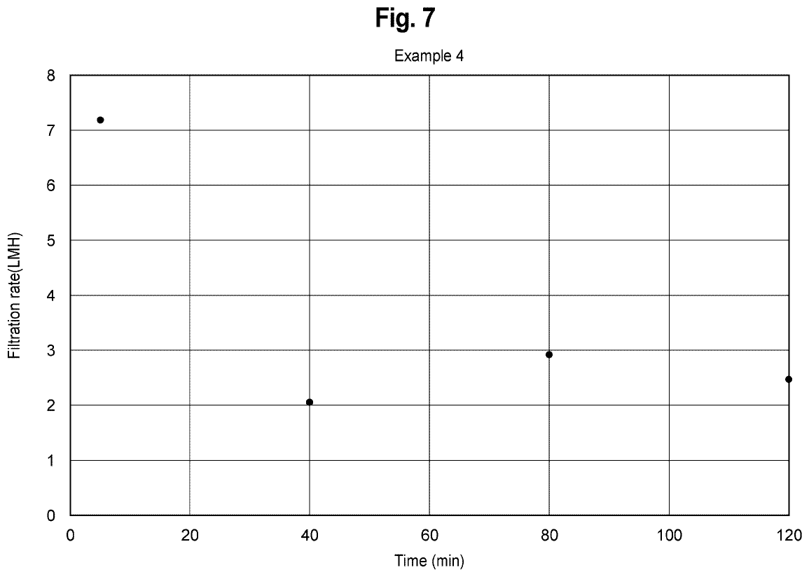

These mAb A-containing solutions were filtered through VF B, and LMH was measured in the same manner as in (6) of Example 1 to obtain the results shown in FIGS. 5 to 7. The flux decay was 0% at the antibody concentration of 10.0 mg/ml, 50.0% at the antibody concentration of 30.0 mg/mL, and 75.0% at the antibody concentration of 50.0 mg/mL. In general, a higher antibody concentration in a solution facilitates clogging due to antibody-antibody association or antibody adsorption to a membrane. Nonetheless, VF B compared with VF A was found to undergo small clogging even for such solutions having a high antibody concentration.

### Examples 5 to 7

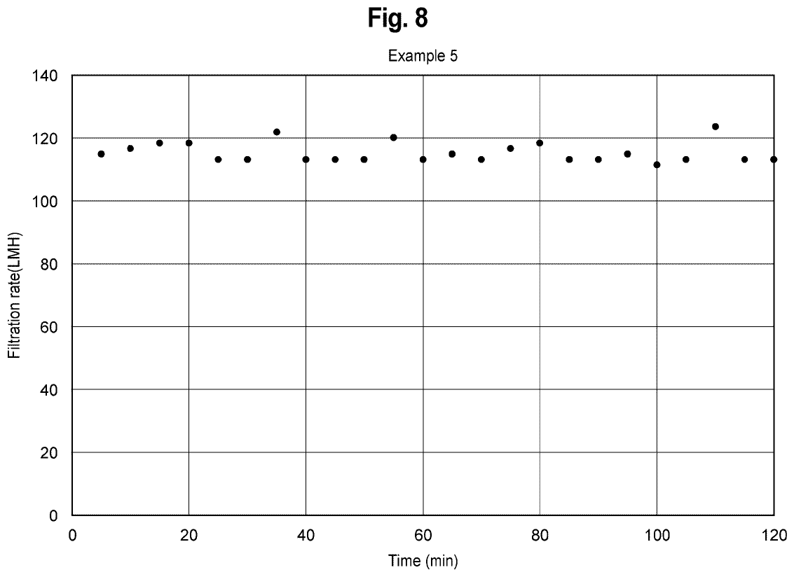

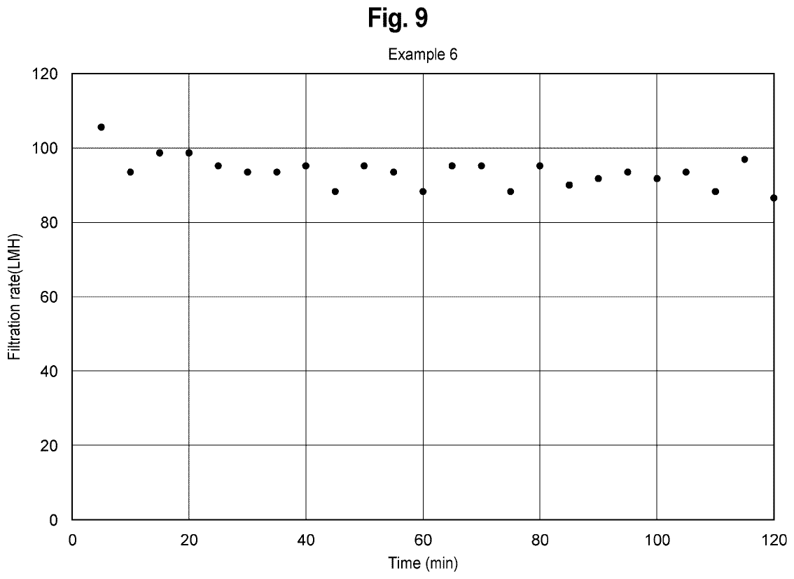

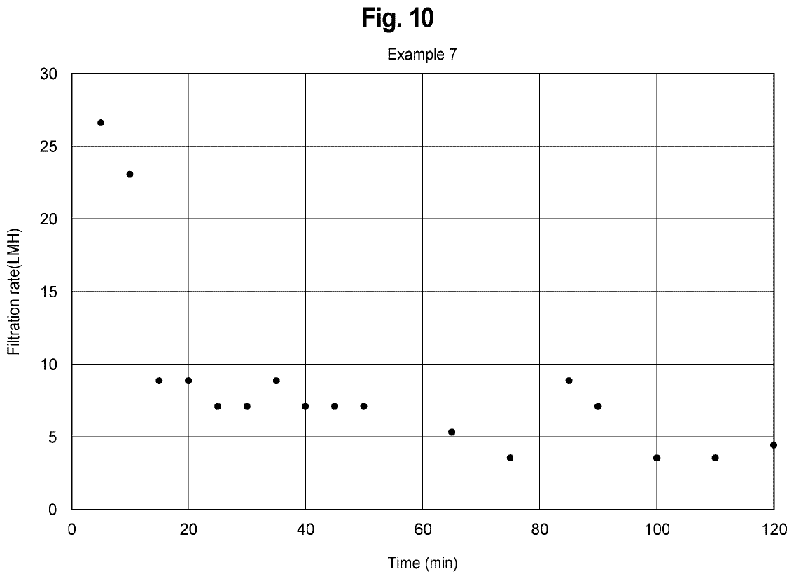

A mAb A-containing solution prepared in the same manner as in (1) and (2) of Example 1 was applied to an antibody concentration, pH, an electric conductivity, and a buffer type changed to the conditions shown in Table 2 to prepare mAb A-containing solutions according to Examples 5 to 7. Each of the mAb A-containing solutions according to Examples 5 to 7 was filtered through VF B, and LMH was measured in the same manner as in (6) of Example 1 to obtain the results shown in FIGS. 8 to 10. The flux decay was 1.5% in the mAb A-containing solution according to Example 5, 18.0% in the mAb A-containing solution according to Example 6, and 83.3% in the mAb A-containing solution according to Example 7. In general, a higher electric conductivity in a solution facilitates clogging due to antibody-antibody association or antibody adsorption to a membrane, while pH of the solution also influences the filtration performance of a filter. Nonetheless, VF B compared with VF A was found to undergo small clogging even at varying pHs and electric conductivities.

### Example 8 and Comparative Example 2

The same experiment as in Example 2 was conducted by changing the antibody type of the antibody solution. A solution containing a monoclonal antibody bevacizumab (manufactured by Pfizer Japan Inc.) was buffer-replaced with a 20 mmol/L acetate buffer solution (pH 5.5, 20 mS/cm) containing sodium chloride to prepare a bevacizumab-containing solution having an antibody concentration of 10.0 mg/mL.

The particle size distribution of particles contained in the bevacizumab-containing solution was measured by DLS in the same manner as in (3) of Example 1. As a result, as shown in FIG. 2, the largest value of the particle size of the particles contained in the bevacizumab-containing solution was 24.4 nm, and the particle size with the largest scattering intensity was 11.7 nm. The particle size distribution of bevacizumab included the range of 22.0 nm or larger. The retention time of bevacizumab in HIC was measured in the same manner as in (4) of Example 1. As a result, the retention time of the highest peak was, as shown in FIG. 3, 19.24 minutes. Thus, Tr−Tm of bevacizumab in hydrophobic interaction chromatography was 15 minutes or more, and the k value thereof was 19 or more.

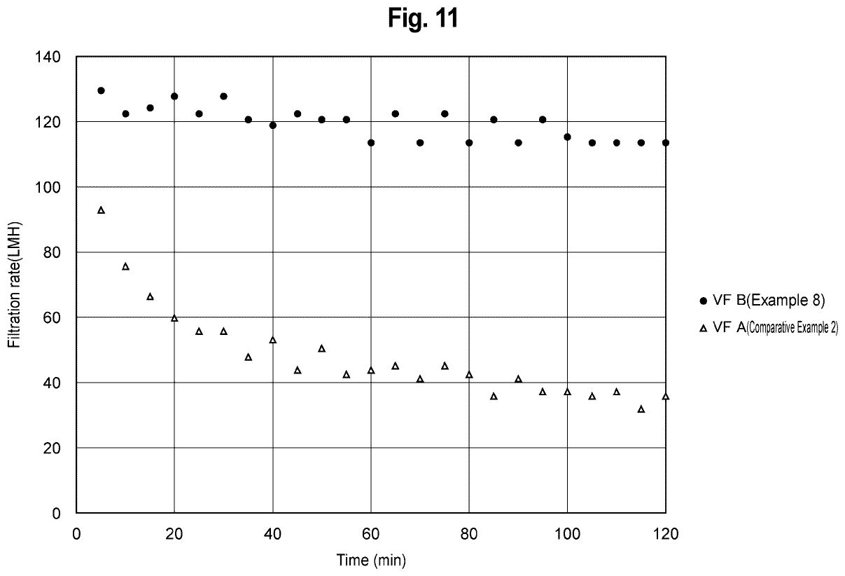

In Comparative Example 2, the bevacizumab-containing solution was filtered through VF A. In Example 8, the bevacizumab-containing solution was filtered through VF B. LMH of Example 8 and Comparative Example 2 was measured in the same manner as in (6) of Example 1 to obtain the results shown in FIG. 11. The flux decay was 61.4% in the case of using VF A according to Comparative Example 2, and 12.3% in the case of using VF B according to Example 8. VF B was found to undergo smaller clogging than that of VF A for bevacizumab, as in the antibody mAb A.

### Example 9 and Comparative Example 3, 4

The same experiment as in Example 2 was conducted by changing the antibody type of the antibody solution. A solution containing a bispecific antibody bimekizumab (manufactured by UCB Japan Co., Ltd.) was buffer-replaced with a 15 mmol/L acetate buffer solution (pH 5.5, 20 mS/cm) containing sodium chloride to prepare a bimekizumab-containing solution having an antibody concentration of 15.4 mg/mL.

The particle size distribution of particles contained in the bimekizumab-containing solution was measured by DLS in the same manner as in (3) of Example 1. As a result, as shown in FIG. 2, the largest value of the particle size of the particles contained in the bimekizumab-containing solution was 24.4 nm, and the particle size with the largest scattering intensity was 13.5 nm. The particle size distribution of bimekizumab included the range of 22.0 nm or larger. The retention time of bimekizumab in HIC was measured in the same manner as in (4) of Example 1. As a result, the retention time of the highest peak was, as shown in FIG. 3, 16.16 minutes. Thus, Tr−Tm of bimekizumab in hydrophobic interaction chromatography was 15 minutes or more, and the k value thereof was 19 or more.

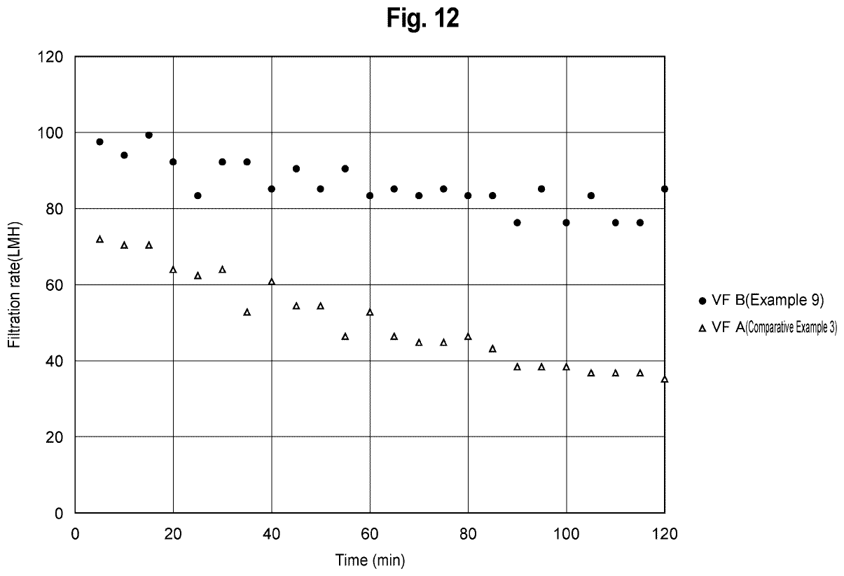

In Comparative Example 3, the bimekizumab-containing solution was filtered through VF A. In Example 9, the bimekizumab-containing solution was filtered through VF B. LMH of Example 9 and Comparative Example 3 was measured in the same manner as in (6) of Example 1 to obtain the results shown in FIG. 12. The flux decay was 51.1% in the case of using VE A according to Comparative Example 3, and 12.7% in the case of using VF B according to Example 9. VF B was found to undergo smaller clogging than that of VF A for bimekizumab, as in the antibodies mAb A and bevacizumab.

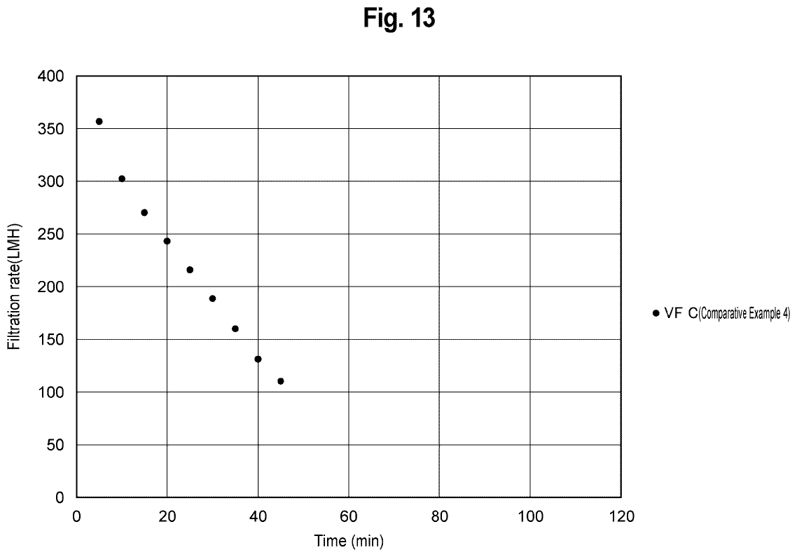

A PES filter VF C was prepared by the method described in Example 1 of International Publication No. WO 2020/203716. In Comparative Example 4, the bimekizumab-containing solution was filtered through VF C. LMH of Comparative Example 4 was measured in the same manner as in (6) of Example 1 to obtain the results shown in FIG. 13. The flux decay was 69.1% in the case of using VF C according to Comparative Example 4. Thus, VF B was found to undergo smaller clogging than that of VF C.

### Comparative Example 5

A culture supernatant containing 2.3 g/L monoclonal antibody mAb B (nivolumab, recombinant human IgG4 monoclonal antibody, molecular weight: approximately 145000) as an antibody protein was provided, and an antibody solution was prepared in the same manner as in (1) and (2) of Example 1 to prepare a mAb B-containing solution having a 15 mmol/L acetate buffer solution (pH 5.5, 15 mS/cm) containing sodium chloride as a liquid, and having an antibody concentration of 14.6 mg/mL.

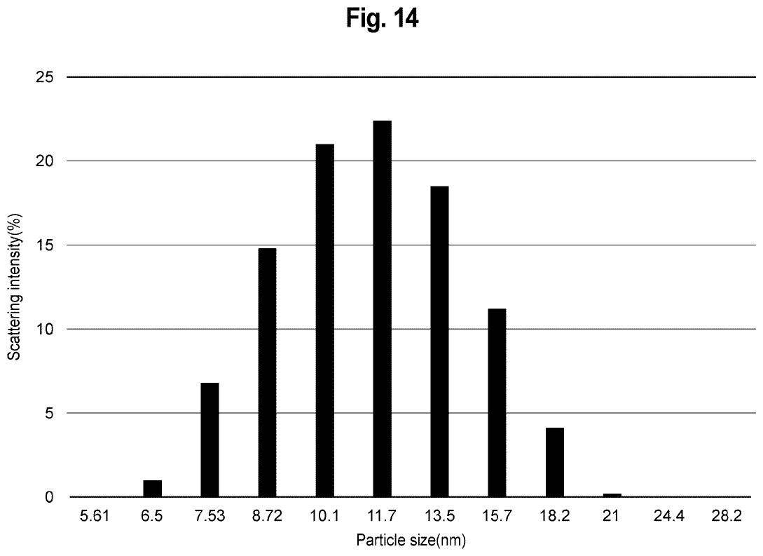

The particle size distribution of particles contained in the mAb B-containing solution was measured by DLS in the same manner as in (3) of Example 1. As a result, as shown in FIG. 14, the largest value of the particle size of the particles contained in the mAb B-containing solution was 21.0 nm, and the particle size with the largest scattering intensity was 11.7 nm. The particle size distribution of mAb B included no range of 22.0 nm or larger. The detection time of mAb B in HIC was measured in the same manner as in (4) of Example 1. As a result, the detection time of the highest peak was, as shown in FIG. 3, 14.46 minutes. Thus, Tr−Tm of the antibody mAb B in hydrophobic interaction chromatography was less than 15 minutes, and the k value thereof was less than 19.

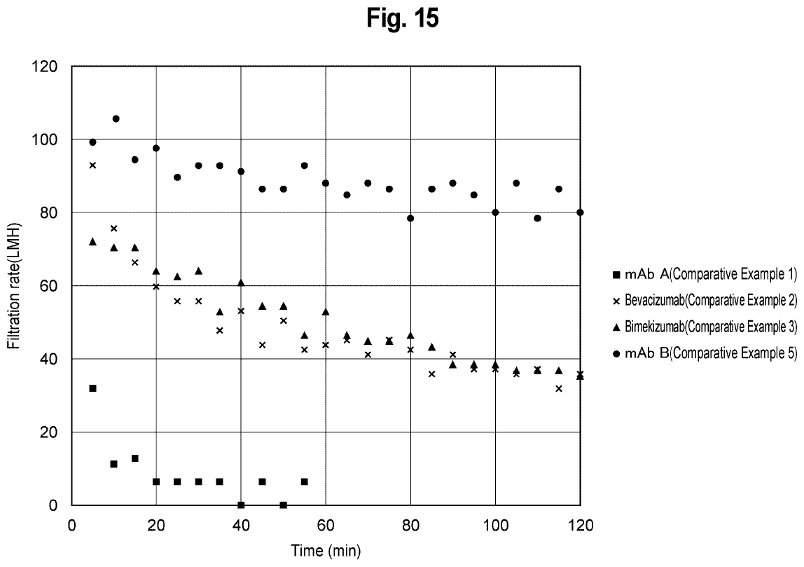

In Comparative Example 5, the mAb B-containing solution was filtered through VF A. LMH of Comparative Example 5 was measured in the same manner as in (6) of Example 1 to obtain the results shown in FIG. 15. The flux decay was 19.4% in the mAb B-containing solution according to Comparative Example 5, and clogging at the same level as in mAb A according to Comparative Example 1, bevacizumab according to Comparative Example 2, and bimekizumab according to Comparative Example 3 did not occur. Thus, an antibody was found unlikely to clog even VF A, wherein the particle size distribution of the antibody included no range of 22.0 nm or larger, or Tr−Tm and the k value of the antibody were less than 15 minutes and less than 19, respectively, in hydrophobic interaction chromatography.

Table 3 shows the DLS measurement results about the antibodies according to Comparative Examples 1 to 4, which largely clogged VF A, and the antibody according to Comparative Example 5, which less clogged VF A. The largest particle sizes in the particle size distributions of the antibodies according to Comparative Examples 1 to 4 were 24.4 nm or larger, whereas the largest particle size in the particle size distribution of the antibody according to Comparative Example 5 was 21.0 nm. In DLS, the association between particles is reflected by the particle size to be measured. The results shown in Table 3 indicate that the antibodies that largely clogged VF A are antibodies that easily undergo antibody-antibody association. In general, a multispecific antibody has an asymmetric charge and degree of hydrophobicity between antigen binding sites and is considered to easily undergo antibody-antibody association as compared with a monoclonal antibody. However, VF B had a low flux decay even for a solution containing an easy-to-associate antibody, indicating that VF B is suitable for the filtration of the solution containing an easy-to-associate antibody.

Table 4 shows results of comparing a Tr−Tm value and a k value among mAb A, bevacizumab, bimekizumab, and mAb B. mAb B which less clogged VF A had a Tr−Tm value of 14.24, whereas mAb A, bevacizumab, and bimekizumab which largely clogged VF A had a Tr−Tm value of 18.90, 19.02, and 16.14, respectively, which were larger than the Tr−Tm value of mAb B. mAb B which less clogged VF A had k=18.3, whereas mAb A, bevacizumab, and bimekizumab which largely clogged VF A had a k value of 24.5, 24.6, and 20.6, respectively, which were larger than the k value of mAb B. Thus, mAb A, bevacizumab, and bimekizumab compared with mAb B were found to have high hydrophobicity. In general, higher hydrophobicity of an antibody facilitates clogging due to adsorption to a filter. Nonetheless, VF B was found insusceptible to the degree of hydrophobicity of an antibody and unlikely to be clogged, because VF B was made of highly hydrophilic cellulose and had an elastic limit pressure as high as 200 kPa or higher.

TABLE 2

| | Example 1 | Example 2 | Example 3 | Example 4 | Example 5 |
|---|---|---|---|---|---|
| VF | VF B | VF B | VF B | VF B | VF B |
| VF material | Regenerated | Regenerated | Regenerated | Regenerated | Regenerated |
| | cellulose | cellulose | cellulose | cellulose | cellulose |
| Antibody type | mAb A | mAb A | mAb A | mAb A | mAb A |
| Antibody concentration | 15 | 10 | 30 | 50 | 10 |
| (mg/mL) | | | | | |
| pH | 5.0 | 5.5 | 5.5 | 5.5 | 5.5 |
| Salt concentration | 200 | 200 | 200 | 200 | 25 |
| (mM, NaCl) | | | | | |
| Electric conductivity | 22 | 20 | 20 | 20 | 4 |
| (mS/cm) | | | | | |
| Buffer type | acetate | acetate | acetate | acetate | acetate |
| Flux decay (%, 120 min) | 28.6~ | 0 | 50.0 | 75.0 | 1.5 |

| | Example 6 | Example 7 | Example 8 | Example 9 |
|---|---|---|---|---|
| VF | VF B | VF B | VF B | VF B |
| VF material | Regenerated | Regenerated | Regenerated | Regenerated |
| | cellulose | cellulose | cellulose | cellulose |
| Antibody type | mAb A | mAb A | Bevacizumab | Bimekizumab |
| Antibody concentration | 10 | 15 | 10 | 15 |
| (mg/mL) | | | | |
| pH | 7.0 | 5.0 | 5.5 | 5.0 |
| Salt concentration | 25 | 700 | 200 | 200 |
| (mM, NaCl) | | | | |
| Electric conductivity | 5 | 56 | 20 | 22 |
| (mS/cm) | | | | |
| Buffer type | tris | acetate | acetate | acetate |
| Flux decay (%, 120 min) | 18.0 | 83.3 | 12.3 | 12.7 |

| | Comparative | Comparative | Comparative | Comparative | Comparative |
|---|---|---|---|---|---|
| | Example 1 | Example 2 | Example 3 | Example 4 | Example 5 |
| VF | VF A | VF A | VF A | VF C | VF A |
| VF material | PVDF | PVDF | PVDF | PES | PVDF |
| Antibody type | mAb A | Bevacizumab | Bimekizumab | Bimekizumab | mAb B |
| Antibody concentration | 15 | 10 | 15 | 15 | 15 |
| (mg/mL) | | | | | |
| pH | 5.0 | 5.5 | 5.0 | 5.0 | 5.5 |
| Salt concentration | 200 | 200 | 200 | 200 | 200 |
| (mM, NaCl) | | | | | |
| Electric conductivity | 22 | 20 | 22 | 22 | 15 |
| (mS/cm) | | | | | |
| Buffer type | acetate | acetate | acetate | acetate | acetate |
| Flux decay (%, 120 min) | 90~ | 61.4 | 51.1 | 69.1 | 19.4 |

TABLE 3

| | Example 1 | Example 8 | Example 9 | |
|---|---|---|---|---|
| | Compar- | Compar- | Compar- | Compar- |
| | ative | ative | ative | ative |
| | Example 1 | Example 2 | Example 3 | Example 5 |
| Antibody type | mAb A | Bevacizumab | Bimekizumab | mAb B |
| Antibody | 15 | 10 | 15 | 15 |
| concentration | | | | |
| (mg/mL) | | | | |
| pH | 5.0 | 5.5 | 5.0 | 5.5 |
| Salt | 200 | 200 | 200 | 200 |
| concentration | | | | |
| (mM, NaCl) | | | | |
| Electric | 22 | 20 | 22 | 15 |
| conductivity | | | | |
| (mS/cm) | | | | |
| Buffer type | acetate | acetate | acetate | acetate |
| DLS peak top | 13.5 | 11.7 | 13.5 | 11.7 |
| (nm) | | | | |
| DLS largest | 28.2 | 24.4 | 24.4 | 21.0 |
| particle size | | | | |
| (nm) | | | | |

TABLE 4

| | Example 1 | Example 8 | Example 9 | |
|---|---|---|---|---|
| | Comparative | Comparative | Comparative | Comparative |
| | Example 1 | Example 2 | Example 3 | Example 5 |
| Antibody | mAb A | Bevacizumab | Bimekizumab | mAb B |
| type | | | | |
| Tr (min) | 19.12 | 19.24 | 16.16 | 14.46 |
| Tr − | 18.90 | 19.02 | 16.14 | 14.24 |
| Tm (min) | | | | |
| k value | 24.5 | 24.6 | 20.6 | 18.3 |

1-20. (canceled)

21. A method for purifying an antibody, comprising:
providing a solution containing the antibody; and
filtering the solution using a porous membrane comprising regenerated cellulose to purify the antibody, wherein
(A) Tr−Tm of the antibody in hydrophobic interaction chromatography is 15 minutes or more, and/or (B) a k value of the antibody in hydrophobic interaction chromatography is 19 or more.

22. The method for purifying an antibody according to claim 21, wherein the porous membrane is a porous hollow fiber membrane.

23. The method for purifying an antibody according to claim 21, wherein an elastic limit pressure of the porous membrane is 200 kPa or higher.

24. The method for purifying an antibody according to claim 21, wherein the Tr−Tm is 16 minutes or more, and/or the k value is 20 or more.

25. The method for purifying an antibody according to claim 21, wherein the antibody is an antibody capable of binding to two or more different antigens.

26. The method for purifying an antibody according to claim 21, wherein the antibody is a monoclonal antibody.

27. The method for purifying an antibody according to claim 21, wherein the antibody is contained in an antibody-drug conjugate.

28. The method for purifying an antibody according to claim 22, wherein a ratio (R/t) of an inner diameter (R) to a membrane thickness (t) of the porous hollow fiber membrane is 8.4 or less.

29. The method for purifying an antibody according to claim 22, wherein a membrane thickness (t) of the porous hollow fiber membrane is in a range of 20 μm or larger and 70 μm or smaller.

30. The method for purifying an antibody according to claim 21, wherein the regenerated cellulose is regenerated cellulose produced by a cuprammonium method.

31. The method for purifying an antibody according to claim 22, wherein a pore size on an inner surface of the porous hollow fiber membrane is larger than that on an outer surface thereof.

32. The method for purifying an antibody according to claim 22, wherein the porous hollow fiber membrane has a gradient structure where a pore size becomes smaller from an inner surface side toward an outer surface side.

33. The method for purifying an antibody according to claim 22, wherein a water permeability of the porous hollow fiber membrane at a filtration pressure of 27 kPa and 37° C. is 10 L/(m2·hr) or more and 50 L/(m2·hr) or less.

34. The method for purifying an antibody according to claim 22, wherein a bubble point of the porous hollow fiber membrane is 1.2 MPa or more.

35. The method for purifying an antibody according to claim 21, wherein a virus is removed in purifying the antibody.

36. The method for purifying an antibody according to claim 35, wherein a parvovirus removal rate (LRV) of the porous membrane is 4.0 or more.

37. The method for purifying an antibody according to claim 21, wherein pH of the solution containing the antibody is 4.5 or higher and 10.0 or lower.

38. The method for purifying an antibody according to claim 21, wherein a salt concentration of the solution containing the antibody is 0 mmol/L or higher and 1000 mmol/L or lower.

39. The method for purifying an antibody according to claim 21, wherein an electric conductivity of the solution containing the antibody is 0 mS/cm or more and 100 mS/cm or less.
